(19) United States
(12) Patent Application Publication
(10) Pub. No.: US 2011/0288086 A1
(43) Pub. Date: Nov. 24, 2011

(54) POLYCYCLIC QUINAZOLINES, PREPARATION THEREOF, AND USE THEREOF

(76) Inventors: Jianqi Li, Shanghai (CN); Zixue Zhang, Shanghai (CN); Peng Xie, Shanghai (CN); Qingwei Zhang, Shanghai (CN)

(21) Appl. No.: 13/112,354

(22) Filed: May 20, 2011

(30) Foreign Application Priority Data
May 21, 2010 (CN) CN 201010182895.7

(51) Int. Cl. A61K 31/535; A61P 35/00; A61K 31/4985; C07D 487/04; A61K 31/519

(52) U.S. Cl. 514/233.2; 544/247; 514/257; 544/115; 514/252.17

(57) ABSTRACT

At least one active pharmaceutical ingredient is chosen from polycyclic quinazolines of formula V,

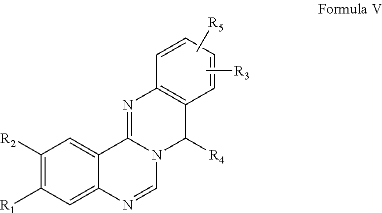

pharmaceutically acceptable salts thereof, and hydrates of the pharmaceutically acceptable salts. The active pharmaceutical ingredients disclosed may be inhibitors of protein tyrosine kinase inhibitors and/or aurora kinase. The active pharmaceutical ingredients can be used for treating cancers susceptible to treatment with protein tyrosine kinase inhibitors and/or aurora kinase inhibitors.

This application claims the benefit of priority under 35 U.S.C, §119 to Chinese Application No. CN 201010182895.7, filed on May 21, 2010.

This disclosure relates to active pharmaceutical ingredients chosen from polycyclic quinazolines disclosed herein, pharmaceutically acceptable salts thereof, and hydrates of the pharmaceutically acceptable salts, and the preparation and use thereof. The active pharmaceutical ingredients disclosed herein can serve as anti-cancer agents in cancers susceptible to treatment with protein tyrosine kinase inhibitors and/or aurora kinase inhibitors.

The protein tyrosine kinase family, either as transmembrane receptors or as cytoplasmic proteins, is widely involved in cell signaling transduction through phosphorylation and dephosphorylation processes. In the process of carcinogenesis, mutation or over expression of protein tyrosine kinases can transform normal cells into cancer cells, and promote tumor cell mitosis and growth. Therefore, inhibitors of these enzymes can be used to treat cancer. For example, the epidermal growth factor receptor (EGFR) tyrosine kinase family includes 4 main members, ErbB-1, ErbB-2, ErbB-3, and ErbB-4. One or more ErbB receptors are over-expressed in about 60% of known tumors, especially in solid tumors such as lung cancer, breast cancer, pancreatic cancer, prostate cancer, stomach cancer, ovarian cancer, etc. Another example is the vascular endothelial growth factor receptor (VEGFR) family, which includes five main members, three of which (VEGFR-1, VEGFR-2, and VEGFR-3) belong to the tyrosine kinase receptor superfamily. High expression of VEGFR and angiogenesis are prerequisite conditions for tumor growth. Thus, blocking the signal transduction of VEGFR and inhibiting angiogenesis is an attractive strategy for cancer therapy. Therefore, EGFR and VEGFR tyrosine kinase inhibitors can be used for the treatment of cancers in human.

Aurora kinases are an important class of serine/threonine kinases in the cell mitotic regulatory network. The aurora kinase family can be categorized as Aurora A, Aurora B, and Aurora C kinases. They play an important role in regulating mitosis. Abnormal expression of aurora kinases likely interferes with mitosis, which may lead to genetic instability and tumor growth. Some aurora kinase inhibitors, including Hesperadin, ZM447439, MK0457 (also known as VX-680), AZD1152, MLN8054, PHA-739358, etc., have been used in preclinical and phase I clinical trials.

Nine small molecule tyrosine kinase inhibitors are currently marketed as anticancer drugs, including gefitinib, erlotinib hydrochloride, and lapatinib tosylate. The structures of those nine tyrosine kinase inhibitors are shown below:

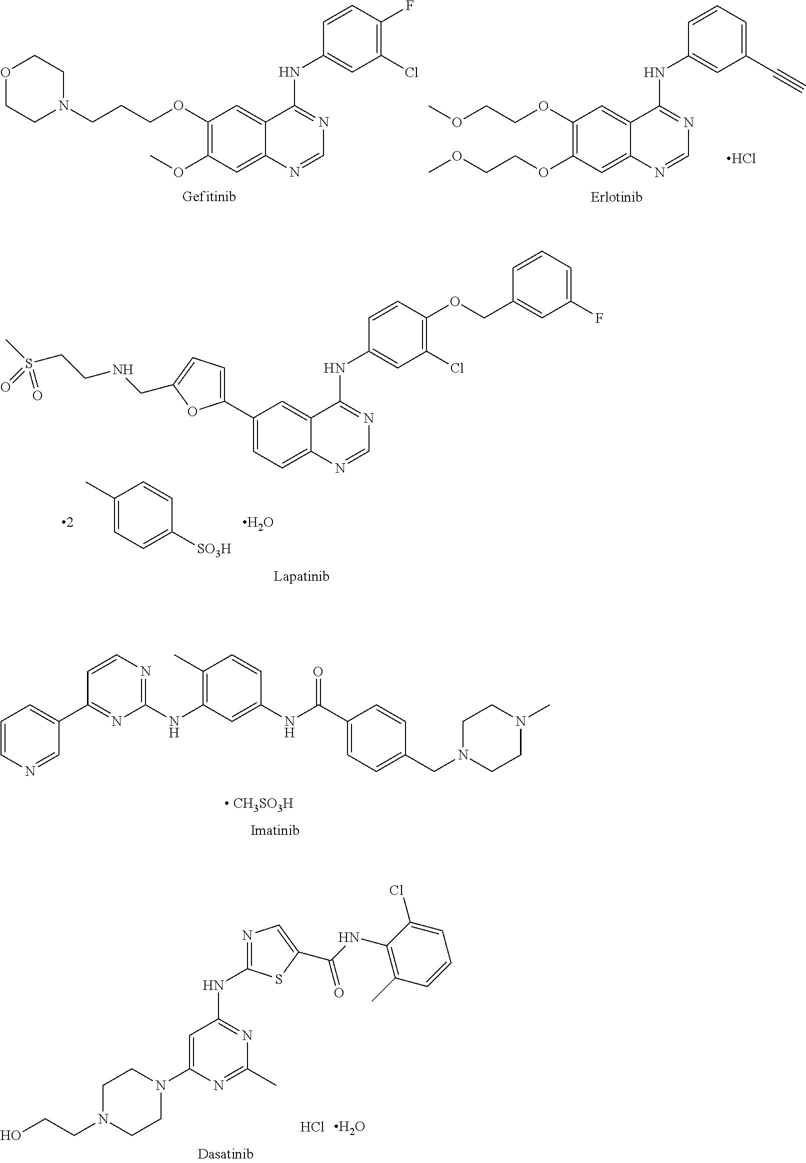

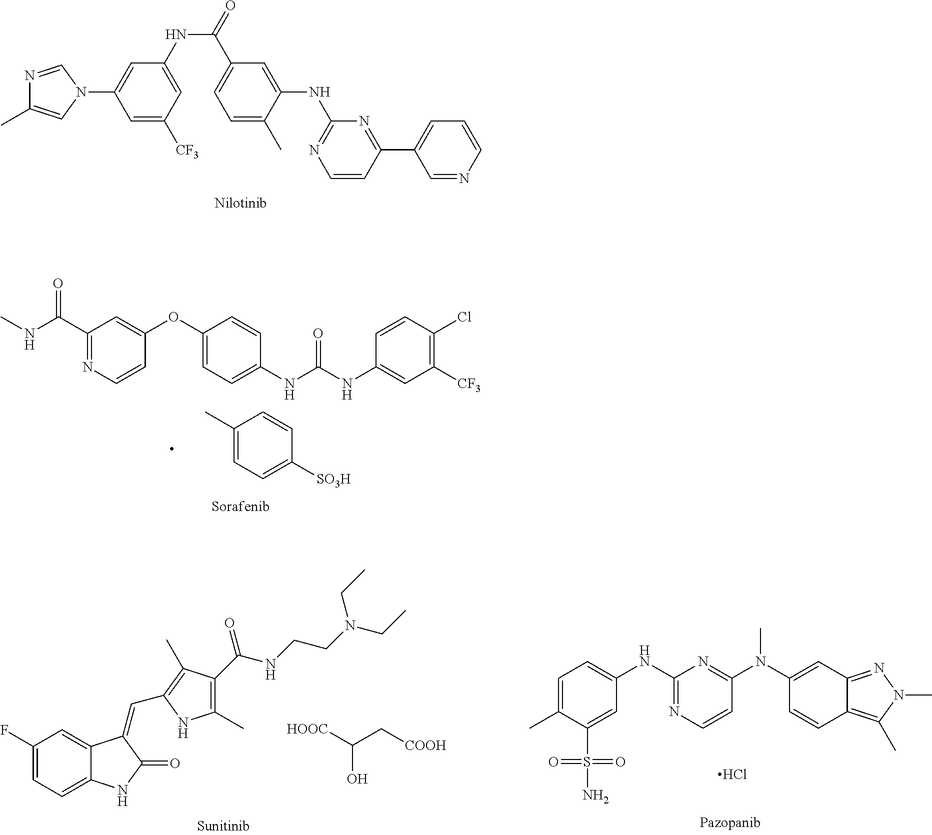

Although the anti-cancer compounds mentioned above have contributed a great deal to the field, research continues in this area to improve anti-cancer drugs.

This disclosure discloses at least one active pharmaceutical ingredient chosen from polycyclic quinazolines of Formula V,

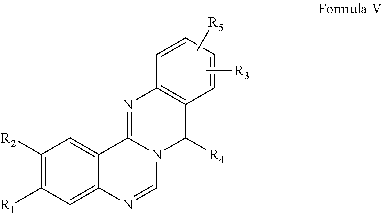

pharmaceutically acceptable salts thereof, and hydrates of the pharmaceutically acceptable salts,
wherein, R₁ and R₂ are independently chosen from hydrogen, halogen, substituted and unsubstituted amino, nitro, carboxyl, substituted and unsubstituted C₁-C₄ alkoxy carbonyl, substituted and unsubstituted C₁-C₄ alkyl, substituted and unsubstituted alkoxy, guanidino, ureido, substituted and unsubstituted C₁-C₄ sulfonyl, substituted and unsubstituted aryl sulfonyl, substituted and unsubstituted aryl, substituted and unsubstituted heterocyclic, and substituted and unsubstituted heteroaryl;
R₃ and R₅ are independently chosen from hydrogen, halogen, cyano, nitro, substituted and unsubstituted amino, hydroxyl, substituted and unsubstituted C₁-C₄ alkyl, substituted and unsubstituted C₁-C₄ alkoxy, substituted and unsubstituted C₂-C₄ unsaturated aliphatic chain, substituted and unsubstituted five-membered aliphatic ring, substituted and unsubstituted six-membered aliphatic ring, substituted and unsubstituted aryl, substituted and unsubstituted heterocyclic, and substituted and unsubstituted heteroaryl;
R₄ is chosen from hydrogen, substituted and unsubstituted C₁-C₄ alkyl, and substituted and unsubstituted C₁-C₄ alkoxy; and
R₁, R₂, R₃, R₄ and R₅ are not simultaneously hydrogen.

In one embodiment, R₁, R₂, R₃, and/or R₅ are independently chosen from substituted and unsubstituted phenyl. For example, the substituted phenyl has 1 to 4 substituents independently chosen from halogen, hydroxyl, nitro, cyano, substituted and unsubstituted C₁-C₄ alkoxy, substituted and unsubstituted C₁-C₄ alkyl, and NH₂.

In one embodiment, R₁, R₂, R₃, and/or R₅ are independently chosen from substituted and unsubstituted benzyloxy. For example, R₁, R₂, R₃, and/or R₅ are independently chosen from 3-F-benzyloxy.

In one embodiment, R₁, R₂, R₃ and/or R₅ are independently chosen from (1) saturated and partially unsaturated, substituted and unsubstituted five-membered heterocyclic; and (2) saturated and partially unsaturated, substituted and unsubstituted six-membered heterocyclic; wherein the substituted and unsubstituted heterocyclic contains at least one hetero atom independently chosen from nitrogen, oxygen, and sulfur.

In one embodiment, R₁ and/or R₂ are independently chosen from hydroxyl amino, substituted and unsubstituted aryl acyl amino, C₁-C₄ acyl amino, and (E)-4-(dimethylamino)-2-butene acylamino.

In one embodiment, R₃ and/or R₅ are independently chosen from C₁-C₄ acyl amino, benzoyl-amino, and C₁-C₄ alkyl amino.

In one embodiment, R₃ and/or R₅ are independently chosen from trifluoromethyl, hydroxymethyl, and alkyl sulfonate.

In one embodiment, R₁, R₂, and/or R₄ are independently chosen from trifluoromethyl.

In one embodiment, R₁, R₂, R₃, and/or R₅ are independently chosen from 5-(((2-(methylsulfonyl)ethyl)amino)methyl)furyl.

In one embodiment, R₁ and/or R₂ are independently chosen from C₁-C₄ alkoxy, benzyloxy, pyrrolidine-1-yl-(C₂-C₄)alkoxy, morpholino-1-yl-(C₂-C₄)alkoxy, piperazin-1-yl-(C₂-C₄)alkoxy, N-methyl piperazine-1-yl-(C₂-C₄) alkoxy, and piperidine-1-yl-(C₂-C₄)alkoxy.

In one embodiment, R₁, R₂, R₃, and/or R₅ are independently chosen from methoxy, 3-F-benzyloxy, pyrrolidine-1-yl-2- propoxy, morpholino-1-yl-2- propoxy, piperazin-1-yl-2- propoxy, N-methyl piperazine-1-yl-2- propoxy, and piperidine-1-yl-2-propoxy.

In one embodiment, the halogen in R₁, R₂, R₃, and/or R₅ is independently chosen from fluorine, chlorine, and bromine.

In one embodiment, the pharmaceutically acceptable salts are chosen from hydrochloride, hydrobromide, sulfate, acetate, lactate, tartrate, tannate, citrate, triflouroacetate, malate, maleate, succinate, p-toluenesulfonate, and mesylate.

In one embodiment, the hydrates of the pharmaceutically acceptable salts have a hydrate number chosen from 0.5, 1, 2, and 3.

In one embodiment, R₁, R₂, R₃, R₄, and/or R₅ are independently chosen from methoxy, ethoxy, n-propoxy, isopropoxy, n-butoxy, isobutoxy, methoxyethyl, 2-methoxy ethoxy, and ethoxy methoxy.

In one embodiment, R₁ and/or R₂ are independently chosen from alkoxy, NH₂, nitro, trifluoromethyl, C₁-C₄ alkyl, C₁-C₄ alkoxy carbonyl, carboxyl, and (E)-4-(dimethylamino)-2-butene acylamino.

In one embodiment, R₁ and/or R₂ are independently chosen from hydrogen, chlorine, n-propyl, NH₂, nitro, trifluoromethyl, methoxy, 2-methoxy-ethoxy, 3-fluoro-benzyloxy, pyrrolidine-1-yl-2- propoxy, morpholino-l-yl-2- propoxy, piperazin-1-yl-2- propoxy, N-methyl piperazine-1-yl-2- propoxy, piperidine-1-yl-2- propoxy, ethoxycarbonyl, carboxyl, 5-(((2-(methylsulfonyl)ethyl)amino)methyl)furyl, and (E)-4-(dimethylamino)-2-butene acylamino.

In one embodiment, R₃ and/or R₅ are independently chosen from hydrogen, halogen, cyano, nitro, NH₂, unsaturated aliphatic chain, alkoxy, hydroxyl, trifluoromethyl, alkyl sulfonate, hydroxymethyl, heterocyclic, and C₁-C₄ acyl amino.

In one embodiment, R₃ and/or R₅ are independently chosen from substituted and unsubstituted C₂-C₄ alkynyl.

In one embodiment, R₃ and/or R₅ are independently chosen from hydrogen, fluorine, chlorine, bromine, cyano, nitro, NH₂, hydroxyl, trifluoromethyl, acetylene, MeS(O)₂O—CH₂— (also known as —CH₂—OMs), MeC(O)NH—, PhC(O)NH—, methoxy, hydroxymethyl, morpholino, and 3-fluoro-benzyloxy.

In one embodiment, R₄ is chosen from hydrogen, methyl, methoxyethyl, trifluoromethyl, and n-butyl.

In one embodiment, at least one polycyclic quinazoline is chosen from:
9-chloro-2,3-bis(2-methoxyethoxy)-8H-quinazolino[4,3-b]quinazoline,
9-bromo-2,3-bis(2-methoxyethoxy)-8H-quinazolino[4,3-b]quinazoline,
9-fluoro-2,3-bis(2-methoxyethoxy)-8H-quinazolino[4,3-b]quinazoline,
9-ethynyl-2,3-bis(2-methoxyethoxy)-8H-quinazolino[4,3-b]quinazoline,
9-nitro-2,3-bis(2-methoxyethoxy)-8H-quinazolino[4,3-b]quinazoline,
9-amino-2,3-bis(2-methoxyethoxy)-8H-quinazolino[4,3-b]quinazoline,
9-cyano-2,3-bis(2-methoxyethoxy)-8H-quinazolino[4,3-b]quinazoline,
9-hydroxy-2,3-bis(2-methoxyethoxy)-8H-quinazolino[4,3-b]quinazoline,
9-trifluoromethyl-2,3-bis(2-methoxyethoxy)-8H-quinazolino[4,3-b]quinazoline,
9-methoxy-2,3-bis(2-methoxyethoxy)-8H-quinazolino[4,3-b]quinazoline,
N-{2,3-bis(2-methoxyethoxy)-8H-quinazolino[4,3-b]quinazoline}acetamide,
N-{2,3-bis(2-methoxyethoxy)-8H-quinazolino[4,3-b]quinazoline}benzamide,
9-chloro-10-fluoro-2,3-bis(2-methoxyethoxy)-8H-quinazolino[4,3-b]quinazoline,
9-chloro-10-(3-fluoro-benzyloxy)-2,3-bis(2-methoxyethoxy)-8H-quinazolino[4,3-b]quinazoline,
11-chloro-2,3-bis(2-methoxyethoxy)-8H-quinazolino[4,3-b]quinazoline,
11-fluoro-2,3-bis(2-methoxyethoxy)-8H-quinazolino[4,3-b]quinazoline,
11-cyano-2,3-bis(2-methoxyethoxy)-8H-quinazolino[4,3-b]quinazoline,
11-hydroxy-2,3-bis(2-methoxyethoxy)-8H-quinazolino[4,3-b]quinazoline,
11-ethynyl-2,3-bis(2-methoxyethoxy)-8H-quinazolino[4,3-b]quinazoline,
mesylate-{9-nitro-2,3-bis(2-methoxyethoxy)-8H-quinazolino[4,3-b]quinazoline-11-methanol}ester,
9-amino-11-hydroxymethyl-2,3-bis(2-methoxyethoxy)-8H-quinazolino[4,3-b]quinazoline,
11-chloro-8-methyl-2,3-bis(2-methoxyethoxy)-8H-quinazolino[4,3-b]quinazoline,
4-(3-((9-chloro-10-fluoro-3-methoxy-8H-quinazolino[4,3-b]quinazoline-2-yl)oxy)propyl)morpholine,
9-chloro-3-methoxy-2-(3-(pyrrolidine-1-yl)propoxy)-8H-quinazolino[4,3-b]quinazoline,
9-fluoro-3-methoxy-2-(3-(piperazinyl-1-yl)propoxy)-8H-quinazolino[4,3-b]quinazoline,
4-(3-((9-ethynyl-3-methoxy-8H-quinazolino[4,3-b]quinazoline-2-yl)oxy)propyl)morpholine,
3-methoxy-2-(3-morpholinyl propoxy)-8H-quinazolino[4,3-b]quinazoline-9-nitrile,
4-(3-((11-chloro-3-methoxy-8H-quinazolino[4,3-b]quinazoline-2-yl)oxy)propyl)morpholine,
mesylate-{3-(((9-nitro--8H-quinazolino[4,3-b]quinazoline-2-yl)oxy)propyl)morpholine-11-methanol}ester,
9-chloro-10-fluoro-2,3-dimethoxy-8H-quinazolino[4,3-b]quinazoline,
9-ethynyl-2,3-dimethoxy-8H-quinazolino[4,3-b]quinazoline,
9-cyano-2,3-dimethoxy-8H-quinazolino[4,3-b]quinazoline,
N-((5-(9-chloro-10-(3-fluoro benzyloxy)-8H-quinazolino[4,3-b]quinazoline-2-yl)furan-2-yl)methyl)-2-(methylsulfonyl)ethylamine,
N-((5-(9-chloro-10-fluoro-8H-quinazolino[4,3-b]quinazoline-2-yl)furan-2-yl)methyl)-2-(methylsulfonyl)ethylamine,
N-((5-(9-ethynyl-8H-quinazolino[4,3-b]quinazoline-2-yl)furan-2-yl)methyl)-2-(methylsulfonyl)ethylamine,
N-((5-(11-chloro-8H-quinazolino[4,3-b]quinazoline-2-yl)furan-2-yl)methyl)-2-(methylsulfonyl)ethylamine,
11-chloro-8-n-butyl-2,3-bis(2-methoxyethoxy)-8H-quinazolino[4,3-b]quinazoline,
11-chloro-2,3-bis(2-methoxyethoxy)-8-(2-methoxyethyl)-8H-quinazolino[4,3-b]quinazoline,
2-chloro-11-cyano-8H-quinazolino[4,3-b]quinazoline,
11-chloro-2-nitro-8H-quinazolino[4,3-b]quinazoline,
11-chloro-2-amino-8H-quinazolino[4,3-b]quinazoline,
11-chloro-2-methoxy-3-trifluoromethyl-8H-quinazolino[4,3-b]quinazoline,
11-chloro-2-(3-fluoro-benzyloxy)3-methoxy-8H-quinazolino[4,3-b]quinazoline,
9-bromo-3-methoxy-2-n-propyl-8H-quinazolino[4,3-b]quinazoline,
9-bromo-8H-quinazolino[4,3-b]quinazoline-2- formic acid ethyl ester,
(E)-(9-bromo-8H-quinazolino[4,3-b]quinazoline-2-yl)-4(dimethylamino)-2-butene amide,
9-bromo-8H-quinazolino[4,3-b]quinazoline-2-formic acid,
4-(2,3-dimethoxy-8H-quinazolino[4,3-b]quinazoline-9-yl)morpholine, and
11-chloro-2,3-dimethoxy-8-trifluoromethyl-8H-quinazolino[4,3-b]quinazoline, pharmaceutically acceptable salts thereof, and hydrates of the pharmaceutically acceptable salts.

In one embodiment, the pharmaceutically acceptable salts are chosen from hydrochloride, hydrobromide, sulfate, acetate, lactate, tartrate, tannate, citrate, trifluoroacetate, malate, maleate, succinate, p-toluenesulfonate, and mesylate.

Also disclosed are pharmaceutical compositions comprising the at least one active pharmaceutical ingredient as disclosed herein and at least one pharmaceutically acceptable carrier.

Also disclosed are methods for treating cancers susceptible to treatment with protein tyrosine kinase inhibitors and/or aurora kinase inhibitors comprising administering to a patient in need thereof an amount effective for treating said susceptible cancers of the at least one active pharmaceutical ingredient.

In one embodiment, the at least one active pharmaceutical ingredient is chosen from:
9-chloro-2,3-bis(2-methoxyethoxy)-8H-quinazolino[4,3-b]quinazoline hydrochloride monohydrate,
9-bromo-2,3-bis(2-methoxyethoxy)-8H-quinazolino[4,3-b]quinazoline hydrochloride hemihydrate,
9-fluoro-2,3-bis(2-methoxyethoxy)-8H-quinazolino[4,3-b]quinazoline mesylate,
9-ethynyl-2,3-bis(2-methoxyethoxy)-8H-quinazolino[4,3-b]quinazoline hydrochloride trihydrate,
9-amino-2,3-bis(2-methoxyethoxy)-8H-quinazolino[4,3-b]quinazoline hydrobromide trihydrate,
mesylate-{9-nitro-2,3-bis(2-methoxyethoxy)-8H-quinazolino[4,3-b]quinazoline-11-methanol}ester hydrochloride,
11-chloro-8-methyl-2,3-bis(2-methoxyethoxy)-8H-quinazolino[4,3-b]quinazoline malate,
4-(3-((9-chloro-10-fluoro-3-methoxy-8H-quinazolino[4,3-b]quinazoline-2-yl)oxy)propyl)morpholine di-p-toluenesulfonate trihydrate, and
(E)-(9-bromo-8H-quinazolino[4,3-b]quinazoline-2-yl)-4(dimethylamino)-2-butene amide hydrochloride.

The polycyclic quinazoline salts disclosed herein may be an anhydrate, or a solvate, such as a hydrate. The solvate may contain less than one, one, or more than one molecule of solvent per molecule of polycyclic quinazoline. For example, the hydrate may contain less than one, one, or more molecules of water per molecule of polycyclic quinazoline. For another example, the hydrate may be a hemihydrate, i.e., containing 1.0 molecule of water per two molecules of polycyclic quinazoline. For another example, the hydrate may contain 0.5-3.0 molecules of water for one molecule of polycyclic quinazoline. The hydrate number as used herein refers to the number of molecules of water per molecule of polycyclic quinazoline in a hydrate. For example, if a hydrate contains two molecules of water (i.e., two equivalents of solvated water) per molecule of polycyclic quinazoline, the hydrate has a hydrate number of 2.0.

Throughout the disclosure, a “substituted” group means one or more hydrogens in the group are replaced with the same number of the substituents. For example, when a substituent is oxo (i.e., ═O), then 2 hydrogens on the atom, where the oxo is attached, are replaced.

For the term “substituted” throughout this disclosure, the “substituent” is chosen from halogen, amino, nitro, hydroxyl, oxo, carbonyl, carboxyl, alkoxy, C₁-C₄ alkoxy carbonyl, C₁-C₄ alkyl, C₂-C₄ unsaturated aliphatic, trimethylfluoro, C₁-C₄ sulfonyl, aryl sulfonyl, 5 or 6-membered aliphatic rings, aryl, and heterocyclic, all of which can be optionally further substituted by these substituents.

The “halogen” as disclosed herein is chosen from fluorine, chlorine, bromine, and iodine.

The “amino” as disclosed herein can be substituted or unsubstituted. The unsubstituted amino is an —NH₂ group. The substituted amino is a group where one or two hydrogen atoms of —NH₂ are independently substituted. For example, one or two hydrogen atoms of —NH₂ can be substituted by alkyl, alkenyl, alkynyl, or alkoxy. For another example, the substituted amino can be chosen from C₁-C₄ alkyl amino, hydroxyl amino, substituted or unsubstituted aryl acyl amino, C₁-C₄ acyl amino, (E)-4-(dimethylamino)-2-butene acylamino, and benzoyl-amino. For another example, the C₁-C₄ alkyl amino can be chosen from methyl amino, ethyl amino, n-propyl amino, isopropyl amino, n-butyl amino, isobutyl amino, and sec-butyl amino. For another example, the substituted amino is chosen from MeC(O)NH— and PhC(O)NH—.

The term “alkoxy” as used herein refers to alkyl ether radical. The alkoxy can be substituted or unsubstituted. For example, the alkoxy can be chosen from substituted and unsubstituted C₁-C₄ alkoxy, substituted and unsubstituted benzyloxy, substituted and unsubstituted pyrrolidine-1-yl-(C₂-C₄)alkoxy, substituted and unsubstituted morpholino-1-yl-(C₂-C₄)alkoxy, substituted and unsubstituted piperazin-1-yl-(C₂-C₄)alkoxy, substituted and unsubstituted N-methyl piperazine-1-yl-(C₂-C₄) alkoxy, and substituted and unsubstituted piperidine-1-yl-(C₂-C₄)alkoxy.

For another example, the alkoxy can be chosen from C₁-C₄ alkoxy, benzyloxy, pyrrolidine-1-yl-(C₂-C₄)alkoxy, morpholino-1-yl-(C₂-C₄)alkoxy, piperazin-1-yl-(C₂-C₄)alkoxy, N-methyl piperazine-1-yl-(C₂-C₄) alkoxy, and piperidine-1-yl-(C₂-C₄)alkoxy, all of which can be further substituted. For example, the alkoxy can be substituted or unsubstituted C₁-C₄ alkoxy.

The “C₁-C₄ alkoxy” as disclosed herein can be chosen from methoxy, ethoxy, n-propoxy, isopropoxy, butoxy, isobutoxy, ethyl methoxy, n-propyl methoxy, isopropyl methoxy, methyl ethoxy, ethyl ethoxy, methyl n-propoxy, methyl isopropoxy, methoxy ethoxy, and ethoxy methoxy. For example, the C₁-C₄ alkoxy can be methoxy or 2-methoxy ethoxy.

For another example, the “(C₂-C₄) alkoxy” as disclosed herein can be chosen from ethoxy, n-propoxy, isopropoxy, butoxy, isobutoxy, ethyl methoxy, n-propyl methoxy, isopropyl methoxy, methyl ethoxy, ethyl ethoxy, methyl n-propoxy, methyl isopropoxy, methoxy ethoxy, and ethoxy methoxy.

For a further example, the “alkoxy” can be chosen from methoxy, 3-F-benzyloxy, pyrrolidine-1-yl-2- propoxy, morpholino-1-yl-2- propoxy, piperazin-1-yl-2-propoxy, N-methyl piperazine-1-yl-2- propoxy, and piperidine-1-yl-2- propoxy.

The “alkyl” as disclosed herein refers to a saturated hydrocarbon chain having a specified number of carbon atoms. The alkyl can be substituted or unsubstituted. For example, C₁-C₄ alkyl refers to a saturated branched, non-branched, or cyclic hydrocarbon chain containing 1 to 4 carbon atoms, where none, one, or more hydrogens can be substituted. For example, the C₁-C₄ alkyl can be chosen from methyl, ethyl, propyl, isopropyl, cyclopropyl, butyl, isobutyl, sec-butyl, tert-butyl, and cyclobutyl. For another example, C₁-C₄ alkyl can be chosen from methoxyethyl, methoxy-n-propyl, methoxy isopropyl, ethoxy methyl, ethoxy ethyl, n-propoxy methyl, and isopropoxy methyl.

The “acyl” as disclosed herein refers to —C(O)—. The “acyl amino” as disclosed herein refers to —C(O)NH—. For example, C₁-C₄ acyl amino can be acetyl amino (CH₃C(O)NH—), propionyl amido (CH₃CH₂C(O)NH—), butyryl amido, or iso-butyl amido. The “aryl acyl amino” refers to a group where the aryl group is connected to the carbonyl group of the acyl amino. For example, the “aryl acyl amino” can be phenyl acyl amino (Ph—C(O)—NH—).

The “sulfonyl” as disclosed herein is —SO₂—. “C₁-C₄ sulfonyl” is a group where “C₁-C₄ alkyl” is connected to “sulfonyl.” For example, C₁-C₄ sulfonyl can be chosen from methyl sulfonyl (denoted as “Ms”), ethyl sulfonyl, propyl sulfonyl, isopropyl sulfonyl, n-butyl sulfonyl, isobutyl sulfonyl, and sec-butyl sulfonyl.

The “aryl” as disclosed herein refers to a carbocyclic aromatic system containing one or more rings wherein such rings may be attached together in a pendent manner or may be fused. For example, an aryl may be chosen from phenyl, naphthyl, anthryl, phenanthryl, and diphenyl.

The “aryl sulfonyl” as used herein means that the “aryl” is connected to the “sulfonyl.”

The “aryl” can be substituted or unsubstituted. For example, the “aryl” can be independently substituted by one, two, three, four, or five substituents. For another example, the “aryl” can be chosen from substituted phenyl. For example, the substituted phenyl may contain 1 to 4 substituents on the phenyl ring, wherein the substituents can be independently chosen from halogen, hydroxyl, nitro, cyano, C₁-C₄ alkoxy, C₁-C₄ alkyl, and NH₂.

The “heterocyclic” as disclosed herein is an aliphatic ring containing one or more, such as one, two, three, and four heteroatoms in the ring, wherein the heteroatom is independently nitrogen, oxygen, or sulfur. The rings may be saturated, or partially unsaturated. For example, the heterocyclic can be a five-membered heterocyclic or a six-membered heterocyclic ring. The heterocyclic may be, for example, tetrahydrofuran, tetrahydropyrrolidine, dihydro-pyrazole, piperidine, piperazine, or morpholino.

The heterocyclic may be substituted or unsubstituted. The substituted heterocyclic means that the hydrogen atoms of the heterocycle can be independently substituted by one or more, e.g., two, three, four, or five, substituents. As used herein, heterocyclic does not overlap with heteroaryl.

The “heteroaryl” as used herein refers to 5- to 12-membered aromatic rings containing one or more, for example, from 1 to 4, or, in some embodiments, from 1 to 3, heteroatoms chosen from N, O, and S, with the remaining ring atoms being carbon. For example, the “heteroaryl” may be a 5 or 6-membered aromatic ring. Examples of 5-membered heteroaryl rings include, but are not limited to, furan, pyrrol, pyrazole, imidazole, isoxazole, thiazole, thiodiazole, tetrazole, and tetrazole. Examples of 6-membered heteroaryl rings include, but are not limited to, pyridine, pyrimidine, and pyridazine.

The heteroaryl may be substituted or unsubstituted. The substituted heteroaryl means that the hydrogen atoms of the heteroaryl can be independently substituted by one or more, e.g., two, three, four, or five, substituents. As used herein, heteroaryl does not overlap with aryl. A substituted heteroaryl may be, for example, 5-(((2-(methylsulfonyl)ethyl)amino)methyl)furyl as shown in the following structure:

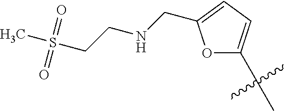

The heteroaryl may contain fused rings. For example, a heteroaryl includes a 5- to 7-membered heteroaromatic ring fused to a 5- to 7-membered aliphatic ring. For such fused, bicyclic heteroaryl ring systems wherein only one of the rings contains one or more heteroatoms, the point of attachment may be at the heteroaromatic ring or the aliphatic ring.

The “unsaturated aliphatic chain” as disclosed herein refers to an unsaturated hydrocarbon having a specified number of carbon atoms. It can be a straight-chain, branched-chain, or cyclic unsaturated hydrocarbon chain. For example, C₂-C₄ unsaturated aliphatic chain is a 2-4 carbon hydrocarbon chain containing at least one double or triple bond. An unsaturated aliphatic chain contains at least one alkenyl and/or alkynyl. The “alkenyl” means a straight chain, branched-chain, or cyclic unsaturated hydrocarbon chain that has a specified number of carbon atoms and that contains at least one double bond —C═C—. For example, (C₂-C₄) alkenyl can be vinyl, propenyl, isopropenyl, butenyl, iso-butyleneyl, or sec-butenyl. The “alkynyl” means a straight chain, branched-chain, or cyclic unsaturated hydrocarbon chain that has a specified number of carbon atoms and that contains at least one triple bond —C≡C—. For example, (C₂-C₄) alkynyl can be chosen from acetylene, propenyl alkynyl, butyl alkynyl, and branched counterparts thereof. The unsaturated aliphatic chain can be substituted or unsubstituted.

The five-membered aliphatic ring as used herein refers to a substituted or unsubstituted five-membered ring. The six-membered aliphatic ring as used herein refers to a substituted or unsubstituted six-membered ring.

The alkyl sulfonate as disclosed herein includes (C₁-C₄ alkyl)-S(O)₂—O—(C₁-C₄)alkyl. For an example, the alkyl sulfonate may be a mesyl group or —CH₂OSO₂Me (depicted also as —CH₂—OMs). An alkyl sulfonate as defined herein can be considered a substituted alkyl.

Unless otherwise specified, a non-specified number for a given substituent as used herein means “one or more”.

In one embodiment, the at least one active pharmaceutical ingredient as disclosed herein is chosen from polycyclic quinazolines as listed below:
V-1 9-chloro-2,3-bis(2-methoxyethoxy)-8H-quinazolino[4,3-b]quinazoline hydrochloride monohydrate,
V-1′ 9-chloro-2,3-bis(2-methoxyethoxy)-8H-quinazolino[4,3-b]quinazoline,
V-2 9-bromo-2,3-bis(2-methoxyethoxy)-8H-quinazolino[4,3-b]quinazoline hydrochloride hemihydrate,
V-2′ 9-bromo-2,3-bis(2-methoxyethoxy)-8H-quinazolino[4,3-b]quinazoline,
V-3 9-fluoro-2,3-bis(2-methoxyethoxy)-8H-quinazolino[4,3-b]quinazoline mesylate,
V-3′ 9-fluoro-2,3-bis(2-methoxyethoxy)-8H-quinazolino[4,3-b]quinazoline,
V-4 9-ethynyl-2,3-bis(2-methoxyethoxy)-8H-quinazolino[4,3-b]quinazoline hydrochloride trihydrate,
V-4′ 9-ethynyl-2,3-bis(2-methoxyethoxy)-8H-quinazolino[4,3-b]quinazoline,
V-5 9-nitro-2,3-bis(2-methoxyethoxy)-8H-quinazolino[4,3-b]quinazoline,
V-6 9-amino-2,3-bis(2-methoxyethoxy)-8H-quinazolino[4,3-b]quinazoline hydrobromide trihydrate,
V-6′ 9-amino-2,3-bis(2-methoxyethoxy)-8H-quinazolino[4,3-b]quinazoline,
V-7 9-cyano-2,3-bis(2-methoxyethoxy)-8H-quinazolino[4,3-b]quinazoline,
V-8 9-hydroxy-2,3-bis(2-methoxyethoxy)-8H-quinazolino[4,3-b]quinazoline,
V-9 9-trifluoromethyl-2,3-bis(2-methoxyethoxy)-8H-quinazolino[4,3-b]quinazoline,
V-10 9-methoxy-2,3-bis(2-methoxyethoxy)-8H-quinazolino[4,3-b]quinazoline,
V-11 N-{2,3-bis(2-methoxyethoxy)-8H-quinazolino[4,3-b]quinazoline}acetamide,
V-12 N-{2,3-bis(2-methoxyethoxy)-8H-quinazolino[4,3-b]quinazoline}benzamide,
V-13 9-chloro-10-fluoro-2,3-bis(2-methoxyethoxy)-8H-quinazolino[4,3-b]quinazoline,
V-14 9-chloro-10-(3-fluoro-benzyloxy)-2,3-bis(2-methoxyethoxy)-8H-quinazolino[4,3-b]quinazoline,
V-15 11-chloro-2,3-bis(2-methoxyethoxy)-8H-quinazolino[4,3-b]quinazoline,
V-16 11-fluoro-2,3-bis(2-methoxyethoxy)-8H-quinazolino[4,3-b]quinazoline,
V-17 11-cyano-2,3-bis(2-methoxyethoxy)-8H-quinazolino[4,3-b]quinazoline,
V-18 11-hydroxy-2,3-bis(2-methoxyethoxy)-8H-quinazolino[4,3-b]quinazoline,
V-19 11-ethynyl-2,3-bis(2-methoxyethoxy)-8H-quinazolino[4,3-b]quinazoline,
V-20 mesylate-{9-nitro-2,3-bis(2-methoxyethoxy)-8H-quinazolino[4,3-b]quinazoline-11-methanol}ester hydrochloride,
V-20′ mesylate-{9-nitro-2,3-bis(2-methoxyethoxy)-8H-quinazolino[4,3-b]quinazoline-11-methanol}ester,
V-21 9-amino-11-hydroxymethyl-2,3-bis(2-methoxyethoxy)-8H-quinazolino[4,3-b]quinazoline,
V-22 11-chloro-8-methyl-2,3-bis(2-methoxyethoxy)-8H-quinazolino[4,3-b]quinazoline malate,
V-22′ 11-chloro-8-methyl-2,3-bis(2-methoxyethoxy)-8H-quinazolino[4,3-b]quinazoline,
V-23 4-(3-((9-chloro-10-fluoro-3-methoxy-8H-quinazolino[4,3-b]quinazoline-2-yl)oxy)propyl)morpholine
V-23′ 4-(3-((9-chloro-10-fluoro-3-methoxy-8H-quinazolino[4,3-b]quinazoline-2-yl)oxy)propyl)morpholine,
V-24 9-chloro-3-methoxy-2-(3-(pyrrolidine-1-yl)propoxy)-8H-quinazolino[4,3-b]quinazoline,
V-25 9-fluoro-3-methoxy-2-(3-(piperazinyl-1-yl)propoxy)-8H-quinazolino[4,3-b]quinazoline,
V-26 4-(3-((9-ethynyl-3-methoxy-8H-quinazolino[4,3-b]quinazoline -2-yl)oxy)propyl)morpholine,
V-27 3-methoxy-2-(3-morpholinyl propoxy)-8H-quinazolino[4,3-b]quinazoline-9-nitrile,
V-28 4-(3-((11-chloro-3-methoxy-8H-quinazolino[4,3-b]quinazoline-2-yl)oxy)propyl)morpholine, V-29 mesylate-{3-(((9-nitro-8H-quinazolino[4,3-b]quinazoline-2-yl)oxy)propyl)morpholine-11-methanol}ester,
V-30 9-chloro-10-fluoro-2,3-dimethoxy-8H-quinazolino[4,3-b]quinazoline,
V-31 9-ethynyl-2,3-dimethoxy-8H-quinazolino[4,3-b]quinazoline,
V-32 9-cyano-2,3-dimethoxy-8H-quinazolino[4,3-b]quinazoline,
V-33 N-((5-(9-chloro-10-(3-fluoro benzyloxy)-8H-quinazolino[4,3-b]quinazoline-2-yl)furan -2-yl)methyl)-2-(methylsulfonyl)ethylamine,
V-34 N-((5-(9-chloro-10-fluoro-8H-quinazolino[4,3-b]quinazoline-2-yl)furan-2-yl)methyl)-2-(methylsulfonyl)ethylamine,
V-35 N-((5-(9-ethynyl-8H-quinazolino[4,3-b]quinazoline-2-yl)furan-2-yl)methyl)-2- (methylsulfonyl)ethylamine,
V-36 N-((5-(11-chloro-8H-quinazolino[4,3-b]quinazoline-2-yl)furan-2-yl)methyl)-2- (methylsulfonyl)ethylamine,
V-37 11-chloro-8-n-butyl-2,3-bis(2-methoxyethoxy)-8H-quinazolino[4,3-b]quinazoline,
V-38 11-chloro-2,3-bis(2-methoxyethoxy)-8-(2-methoxyethyl)-8H-quinazolino[4,3-b]quinazoline,
V-39 2-chloro-11-cyano-8H-quinazolino[4,3-b]quinazoline,
V-40 11-chloro-2-nitro-8H-quinazolino[4,3-b]quinazoline,
V-41 11-chloro-2-amino-8H-quinazolino[4,3-b]quinazoline,
V-42 11-chloro-2-methoxy-3-trifluoromethyl-8H-quinazolino[4,3-b]quinazoline,
V-43 11-chloro-2-(3-fluoro-benzyloxy)3-methoxy-8H-quinazolino[4,3-b]quinazoline,
V-44 9-bromo-3-methoxy-2-n-propyl-8H-quinazolino[4,3-b]quinazoline,
V-45 9-bromo-8H-quinazolino[4,3-b]quinazoline-2- formic acid ethyl ester,
V-46 (E)-(9-bromo-8H-quinazolino[4,3-b]quinazoline-2-yl)-4(dimethylamino)-2-butene amide hydrochloride,
V-46′(E)-(9-bromo-8H-quinazolino[4,3-b]quinazoline-2-yl)-4(dimethylamino)-2-butene amide,
V-47 9-bromo-8H-quinazolino[4,3-b]quinazoline-2-formic acid,
V-48 4-(2,3-dimethoxy-8H-quinazolino[4,3-b]quinazoline-9-yl)morpholine, and
V-49 11-chloro-2,3-dimethoxy-8-trifluoromethyl-8H-quinazolino[4,3-b]quinazoline.

The above listed active pharmaceutical ingredients denoted as V-1 to V-49 are illustrated by chemical structures below:

| Number | Chemical structure |
|---|---|
| V-1 | 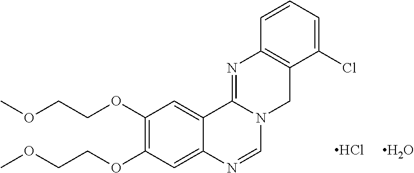 |
| V-2 | 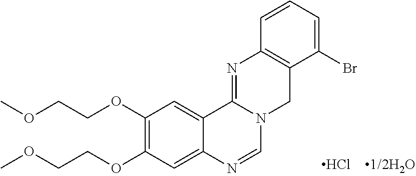 |
| V-3 | 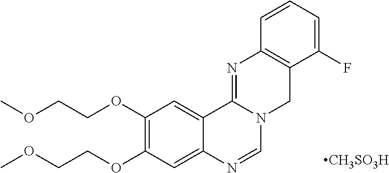 |
| V-4 | 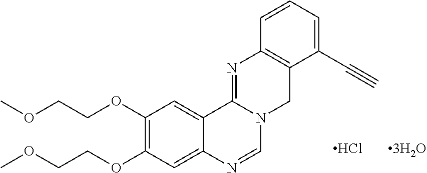 |
| V-5 | 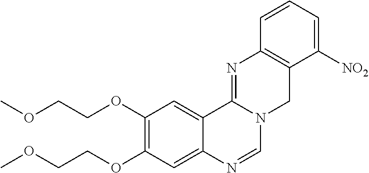 |
| V-6 | 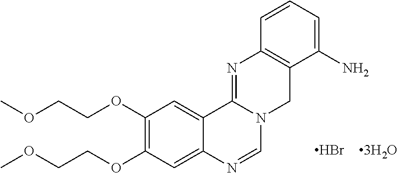 |
| V-7 | 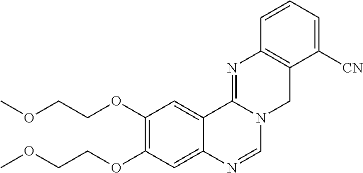 |
| V-8 | 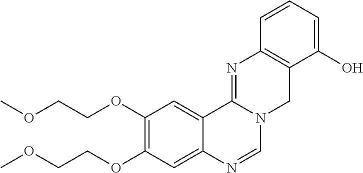 |
| V-9 | 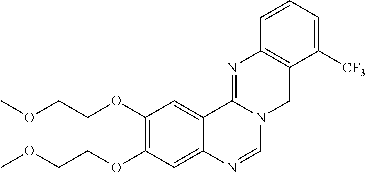 |
| V-10 | 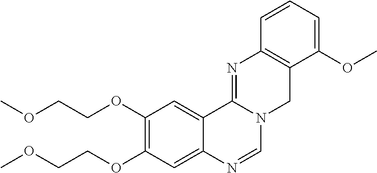 |
| V-11 | 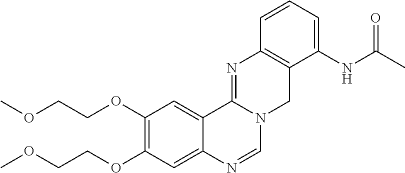 |
| V-12 | 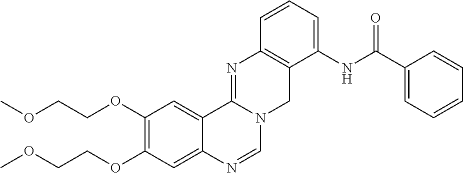 |
| V-13 | 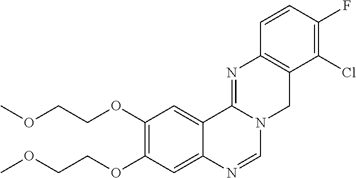 |
| V-14 | 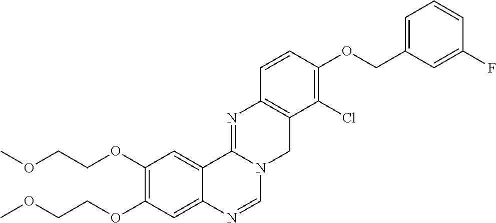 |
| V-15 | 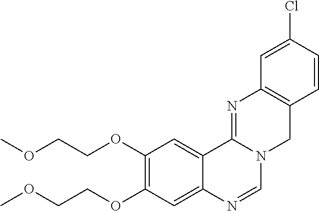 |
| V-16 | 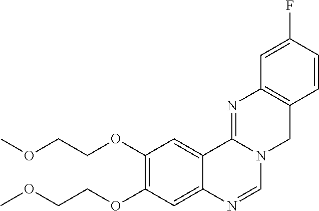 |
| V-17 | 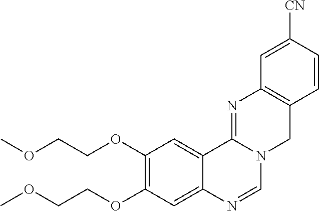 |
| V-18 | 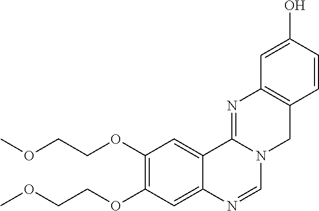 |
| V-19 | 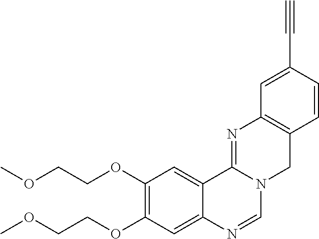 |
| V-20 | 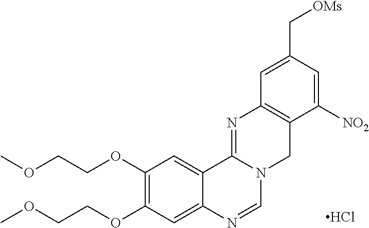 |
| V-21 | 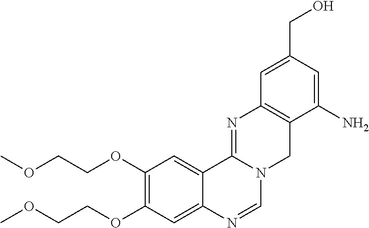 |
| V-22 | 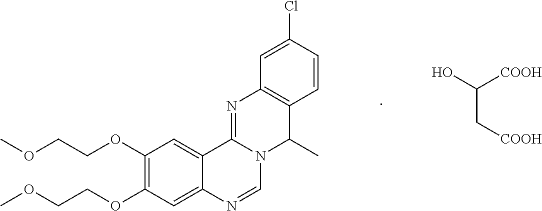 |
| V-23 | 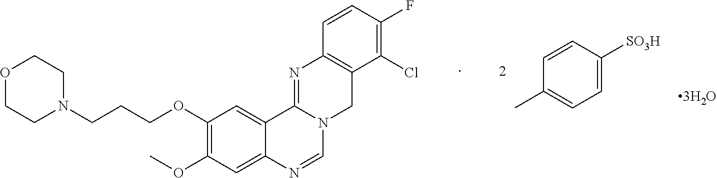 |
| V-24 | 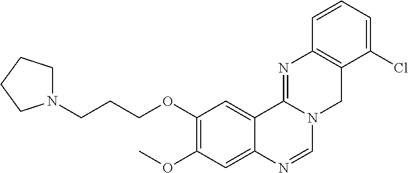 |
| V-25 | 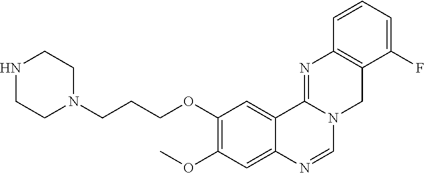 |
| V-26 | 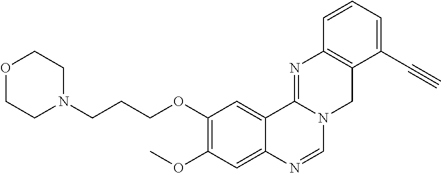 |
| V-27 | 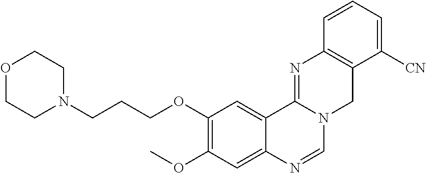 |
| V-28 | 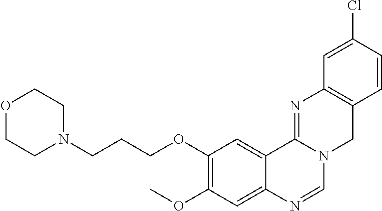 |
| V-29 | 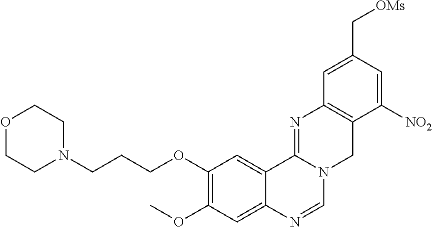 |
| V-30 | 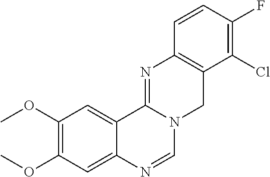 |
| V-31 | 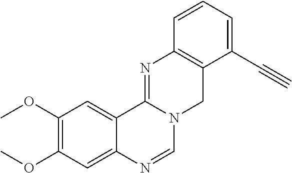 |
| V-32 | 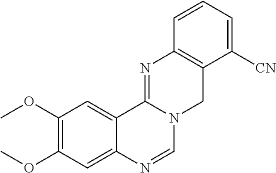 |
| V-33 | 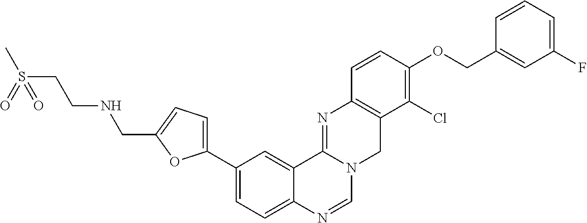 |
| V-34 | 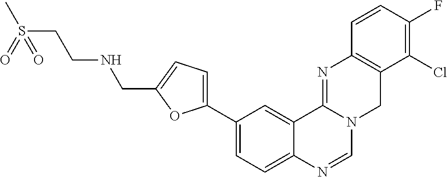 |
| V-35 | 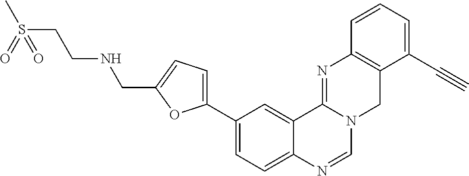 |
| V-36 | 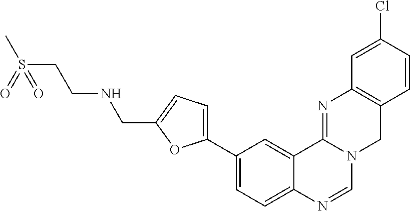 |
| V-37 | 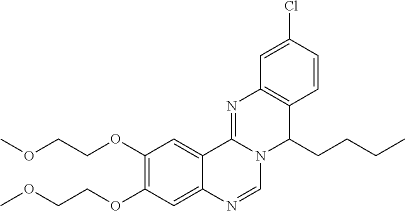 |
| V-38 | 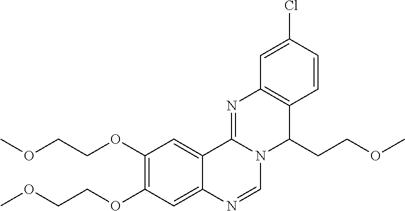 |
| V-39 | 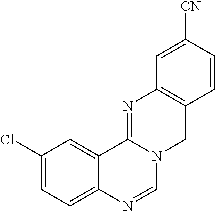 |
| V-40 | 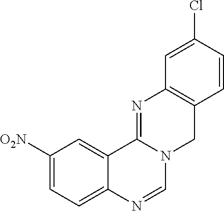 |
| V-41 | 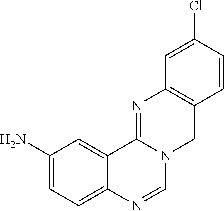 |
| V-42 | 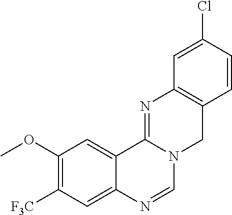 |
| V-43 | 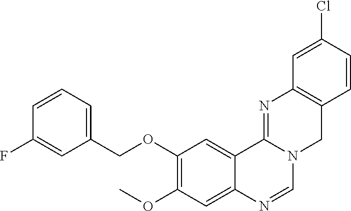 |
| V-44 | 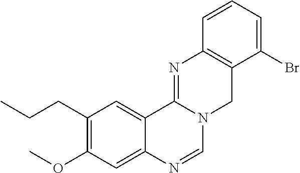 |
| V-45 | 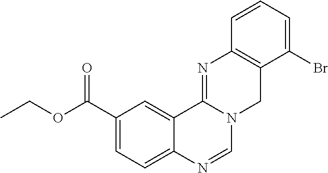 |
| V-46 | 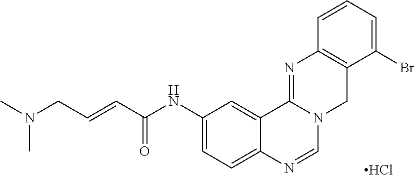 |
| V-47 | 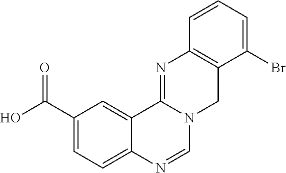 |
| V-48 | 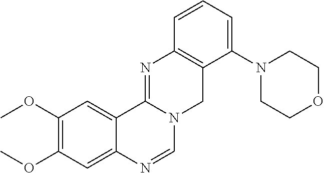 |
| V-49 | 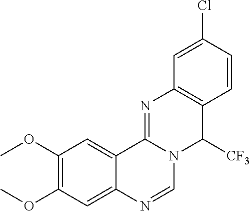 |

The above active pharmaceutical ingredients denoted as V-1′ to V-4′, V-6′, V-20′, V-22′, V-23′, and V-46′ are illustrated by chemical structures below:

| | |
|---|---|
| V-1′ | 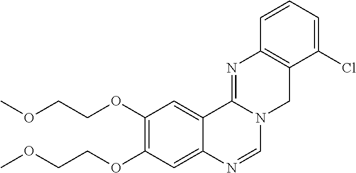 |
| V-2′ | 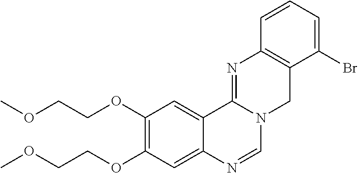 |
| V-3′ | 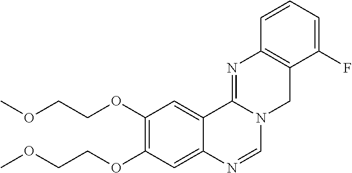 |
| V-4′ | 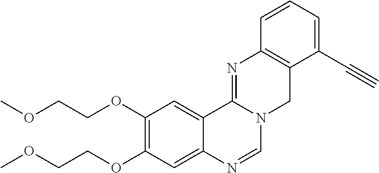 |
| V-6′ | 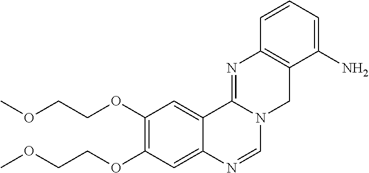 |
| V-20′ | 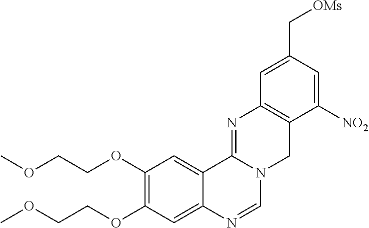 |
| V-22′ | 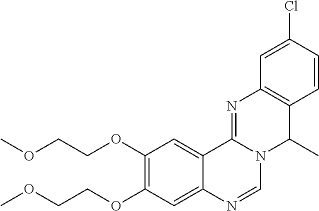 |
| V-23′ | 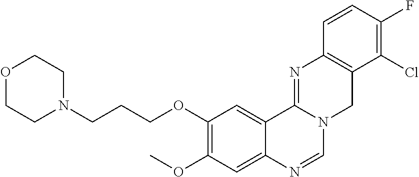 |
| V-46′ | 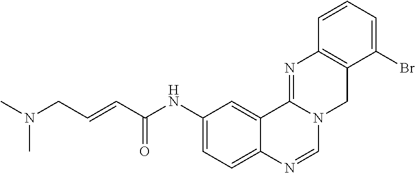 |

The polycyclic quinazolines, salts thereof, and hydrates of the salts can be synthesized by the general and exemplary methods below.

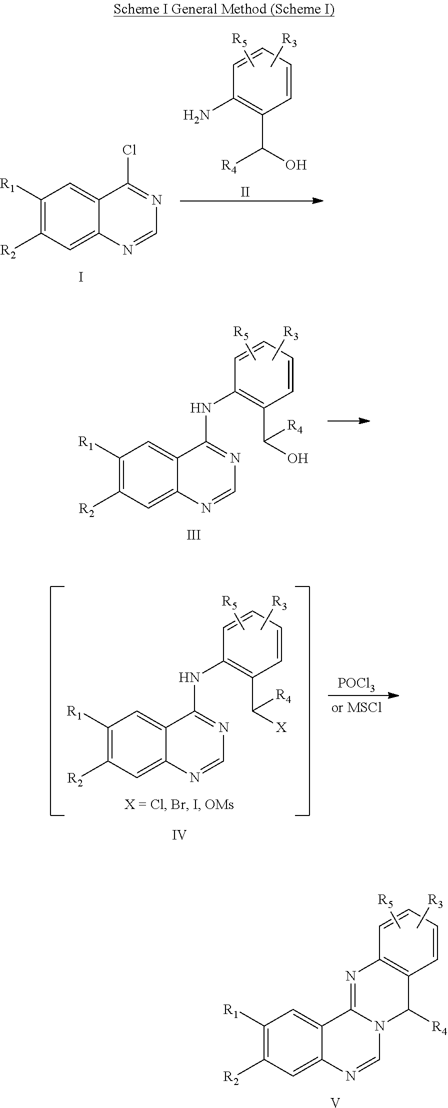

The Reagents, denoted as I (10 mmol) and II (11 mmol) above, are dissolved in solvent A to form a solution, base A (2 mmol) or acid B (2 mmol) is then added dropwise to the solution. The reaction is stopped after stirring for 3-7 h at room temperature. The reaction mixture is filtered to obtain a precipitate. The precipitate is concentrated to obtain intermediate III.

Intermediate III is dissolved in solvent B to obtain a solution. POCl₃ or methanesulfonyl chloride (M_{S}Cl) is added dropwise to the solution. The reaction reacts at a temperature ranging from room temperature to the reflux state over 1-3 h. The unreacted reagents are evaporated and discarded, and the residue is transferred into ice water. The pH of the ice water containing the residue is adjusted to about 10 with saturated Na₂CO₃ solution. The ice water containing the residue is extracted by ethyl acetate three times, and the organic layers are combined. The combined organic layers are washed with water and then with saturated sodium chloride solution. The organic layer is dried by anhydrous sodium sulfate, and filtered to remove anhydrous sodium sulfate. The filtrate is concentrated under vacuum to obtain a brown oil or solid. The brown oil or solid is recrystallized or purified by flash column chromatography to obtain product V. Product V is dissolved in a solution of acid in alcohol solution (3 mol/L) at elevated temperature, and then the solution is cooled down to obtain a precipitate, which is a salt of compound V.

The acid as described above is chosen from hydrochloric acid, hydrobromic acid, sulfuric acid, acetic acid, lactic acid, tartaric acid, tannic acid, citric acid, trifluoroacetic acid, malic acid, maleic acid, succinic acid, p-toluenesulfonic acid, and methanesulfonic acid. Thus the salt obtained is hydrochloride, hydrobromide, sulfate, acetate, lactate, tartrate, tannic acid salt (tannate), citric acid salt (citrate), trifluoroacetic acid salt (trifluoroacetate), malate, maleate, succinate, p-toluenesulfonate, or mesylate.

R₁, R₂, R₃, R₄, and R₅ as shown in Scheme I are defined as in Formula V.

Solvent A as mentioned above can be chosen from acetonitrile, isopropyl alcohol, dichloromethane, N,N-dimethylformamide, N,N-dimethylacetamide, and 1,4-dioxane. Solvent B can be chosen from acetonitrile, dichloromethane, chloroform, toluene, and benzene. Base A can be chosen from triethylamine, pyridine, potassium carbonate, and diisopropyl ethylamine. Acid B can be chosen from formic acid, acetic acid, hydrochloric acid, sulfuric acid, and phosphoric acid.

Reagent I can be synthesized according to the method disclosed in WO patent No. 9630347 and Tetrahedron Letters, 2004 (45), 6517-6521 (see Scheme II). Reagent II can be synthesized according to the method disclosed in J. Med. Chem.1986, 29. 1406-1412 and WO patent No. 2005067933A (see Scheme III).

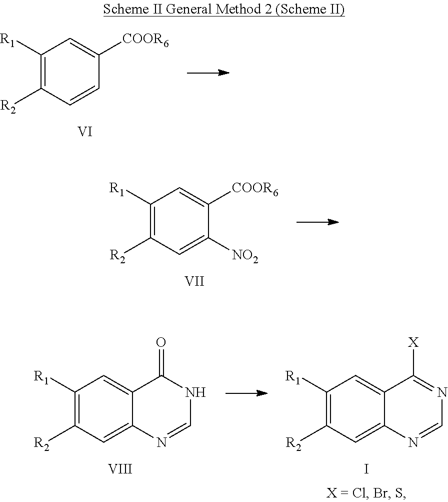

R₁ and R₂ as shown in Scheme II are the same as defined for Formula V. R₆ can be chosen from hydrogen, methyl, ethyl, and isopropyl.

Compound VI (0.05 mol) is dissolved in glacial acetic acid 50 ml. The glacial acetic acid solution is stirred in an ice bath, and 13 ml of nitric acid (65-68% by weight) is added into the glacial acetic acid solution. Then the reaction solution is stirred for 24 h at room temperature. The reaction solution is poured into 500 ml of ice water, and extracted by ethyl acetate three times. The organic phase is combined, washed with saturated NaHSO₄ solution three times, then washed with saturated sodium chloride solution, and dried by anhydrous sodium sulfate. The solution dried by the anhydrous sodium sulfate is then filtered, and the filtrate is concentrated to obtain compound VII.

Compound VII (0.10 mol), ammonium formate (63 g, 1.00 mol), 5% Pd/C (5.00 g), and formamide (75 ml) are combined and reacted at 150° C. for 6.5 h. The reaction mixture is cooled to room temperature and allowed to precipitate. Then the reaction mixture is filtered to obtain a white solid compound VIII.

Compound VIII (0.15 mol) is dissolved in the mixed solution of N,N-dimethylformamide (1 ml) and dichloromethane (150 ml). The solution is stirred at room temperature, while thionyl chloride (14.9 g) (or oxalyl chloride or phosphorus oxychloride) is added dropwise to the solution. The solution is then refluxed for 6 h. The reaction mixture is cooled to room temperature, and the pH value is adjusted to 7-8 with sodium hydroxide solution. The reaction mixture is left to settle. The organic phase of the reaction mixture is collected and concentrated under vacuum to obtain compound I.

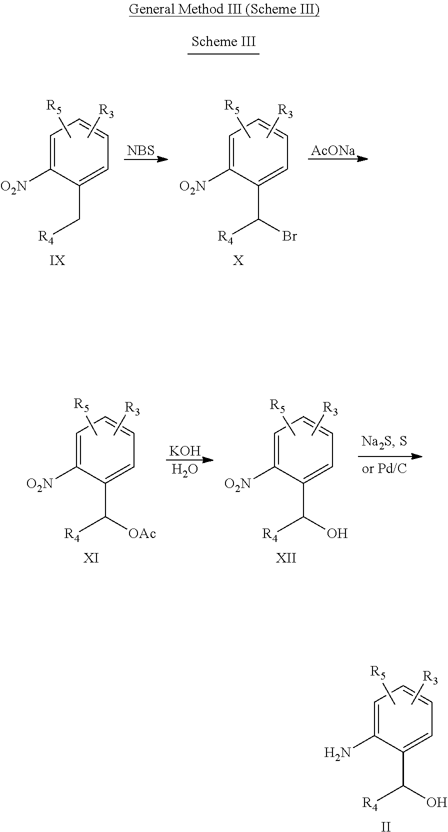

The compound IX (0.20 mol), N-bromosuccinimide (26.6 g, 0.20 mol), and benzoyl peroxide (0.40 g, 2 mmol) are dissolved in 100 ml chlorobenzene (or carbon tetrachloride), heated, and refluxed for 20 h. After the reaction, the solution is filtered to obtain a filtrate. The filtrate is concentrated under vacuum to obtain a light brown oily substance, which is then recrystallized in ethanol or purified by silica gel column chromatography to obtain the compound X.

Compound X (0.05 mol) and anhydrous sodium acetate (14.7 g, 0.15 mol) are added into 70 ml N,N-dimethylformamide, heated, and reacted for 1 h at 70° C. The solution is cooled down to room temperature, 150 ml water is added to the solution, and then the solution is extracted by ethyl acetate three times, washed with water three times, then washed with saturated sodium chloride solution three times, and dried by anhydrous sodium sulfate. The solution is then filtered to obtain a filtrate, which is concentrated under vacuum to obtain a white solid compound XI.

Compound XI (0.01 mol) is added into 100 ml water to form a solution, and 20 ml of potassium hydroxide aqueous solution (10% by weight) is then added dropwise to the solution. The solution is then heated to reflux for 2 h. After the reaction, the reaction mixture is extracted by ethyl acetate three times. The organic phase is combined, washed with water and saturated sodium chloride solution sequentially, and dried by anhydrous sodium sulfate. The reaction mixture is then filtered, and the filtrate is concentrated under vacuum to obtain compound XII.

Compound XII (5 mmol), sodium sulfide (0.78 g, 10 mmol), and sulfur (0.32 g, 10 mmol) are dissolved in the mixed solution of 50 ml ethanol and 25 ml water, then heated to reflux for 2 h. The reaction mixture is cooled down to room temperature, and the ethanol is evaporated under vacuum. The reaction mixture is then added into 30 ml water, and extracted by ethyl acetate. The organic phase is combined, washed with water and saturated sodium chloride solution, and dried by anhydrous sodium sulfate. The reaction mixture is then filtered to obtain a filtrate, which is concentrated under vacuum to obtain compound II.

R₃, R₄, and R₅ as shown in Scheme III are the same as defined for Formula V.

Compounds VI and IX can be purchased through commercial sources or synthesized by conventional methods.

Pharmacological tests have shown that the active pharmaceutical ingredients disclosed herein not only demonstrate inhibition on epidermal growth factor receptor tyrosine kinase and aurora kinase, but also have anti-proliferative activity on certain tumor cells. The active pharmaceutical ingredients disclosed herein can be used for the treatment of cancer and diseases that are susceptible to treatment with protein tyrosine kinase inhibitors and/or aurora kinase inhibitors. For example, the active pharmaceutical ingredients disclosed herein can be used to treat leukemia and/or solid tumors. See Examples 54 and 55.

“Treatment” or “treating” means any treatment of a disease in a patient, including: inhibiting the disease; slowing or arresting the development of at least one clinical symptom of a disease; and relieving the disease, that is, causing the regression of at least one clinical symptom of a disease. “Treating” or “treatment” also refers to inhibiting a disease, either physically, (e.g., stabilization of a discernible symptom), physiologically, (e.g., stabilization of a physical parameter), or both, and inhibit at least one physical parameter which may not be discernible to the subject. Further, “treating” or “treatment” refers to delaying the onset of a disease or at least one clinical symptom thereof in a subject which may be exposed to or predisposed to a disease even though the subject does not yet experience or display symptoms of the disease.

The disclosure also relates to a composition comprising a therapeutically effective amount of the at least one active pharmaceutical ingredient as described herein and at least one pharmaceutically acceptable carrier. The at least one pharmaceutically acceptable carrier may be chosen from bespices, sweeteners, liquid, solid filler, diluent, and other commonly used carrier material. The composition can be made into a common pharmaceutical dosage form by using methods known in the field, such as tablets, capsules, powder, syrup, liquid, suspension, or injection. The weight percentage of the at least one active pharmaceutical ingredient in the composition may range from about 1% to about 70%, for example, from about 5% to about 50%.

The at least one active pharmaceutical ingredient disclosed herein can be administered to mammals (included humans) by oral administration or injection. For example, the at least one active pharmaceutical ingredient can be orally administered. Daily dosage can range from 0.0001 to 200 mg/kg by body weight. The optimal dosage may depend on the individual physiological condition. For example, the administration can start with a lower dosage, and then the dosage can gradually increase.

As shown by animal experiments in Example 56 below, the at least one active pharmaceutical ingredient disclosed herein has very low toxicity.

The present disclosure is illustrated further with the following non-limiting examples. Percentage as mentioned below means weight percentages unless otherwise specified.

### EXAMPLE 1

### 9-chloro-2,3-bis(2-methoxyethoxy)-8H-quinazolino[4,3-b]quinazolinehydrochloride monohydrate(V-1)

4-chloro-6,7-bis(2-methoxyethoxy)quinazoline(3.12 g, 10 mmol) (synthesized according to the General Method II) and 2-amino-6-chloro-benzyl alcohol (1.57 g, 10 mmol) (synthesized according to General Method Ill) were dissolved in 20 ml isopropanol, and heated to reflux for about 4 h. A white solid precipitated. The precipitate was filtered to obtain the crude product, which was recrystallized by ethanol and dried to obtain a white solid intermediate M-1 (4.11 g, yield 87.63%).

M-1 (2.00 g, 4.26 mmol) was dissolved in 3 ml POCl₃, and heated to reflux for about 2 h. The unreacted POCl₃ was evaporated, and the residue was added into ice water, the pH of the mixture was adjusted to 10 by saturated sodium carbonate (Na₂CO₃) solution, and the mixture was extracted by ethyl acetate three times. The organic phase was combined, washed with water and with saturated sodium chloride (NaCl) solution, and dried by anhydrous sodium sulfate. The organic phase was filtered, and the filtrate was concentrated under vacuum to obtain a crude product as a light yellow solid. The crude product was purified by column chromatography to obtain a white solid V-1′ (1.33 g, 72.28%).

The white solid V-1′ obtained above was dissolved in 3 mol/L hydrochloric acid/ethanol (10 ml) and heated until it was fully dissolved. It was then cooled and precipitated to obtain 1.2 g of the compound V-1, which contains 1 equivalent of solvated water as shown by elemental analysis.

MS(ESI): [M+H]⁺=416.

¹H-NMR (400 MHz,CDCl₃) δppm:3.50(s,6H), 3.82(m,4H), 4.22(t,J=8.4 Hz,2H), 4.30(t,J=8.4 Hz2H), 5.59(s,2H), 7.10(s,1H), 7.21(m,2H), 7.39(m, 1H), 7.90(s,1H), 8.40(s,1H).

### EXAMPLE 2

### 9-bromo-2,3-bis(2-methoxyethoxy)-8H-quinazolino[4,3-b]quinazoline hydrochloride hemihydrate(V-2)

4-chloro-6,7-bis(2-methoxyethoxy)quinazoline (3.12 g, 10 mmol) and 2-amino-6-bromo-benzyl alcohol (2.01 g, 10 mmol) (synthesized according to General Method III) were dissolved in 20 ml acetonitrile to form a solution, and 0.2 ml pyridine was added dropwise to the solution. The reaction mixture was heated to reflux for about 4 h, and then a white solid precipitated. The white solid was filtered to obtain a crude product, which was then recrystallized by ethanol and dried to obtain a white solid intermediate M-2 (4.56 g, 88.89%).

M-2 (2.00 g, 3.90 mmol) was dissolved in 3 ml POCl₃, and heated to reflux for about 2 h. The unreacted POCl₃ was evaporated, and the residue was added into ice water. The pH was adjusted to about 9 by saturated sodium carbonate (Na₂CO₃) aqueous solution. The residue was extracted by ethyl acetate three times. The organic phase was combined, washed with water and saturated sodium chloride (NaCl) solution, and dried by anhydrous sodium sulfate. The organic phase was then filtered to obtain a filtrate, which was concentrated under vacuum to obtain a light yellow solid crude product, which was separated by column chromatography to obtain a grayish white solid V-2′ (1.21 g, 67.59%).

The grayish white solid V-2′ obtained above was dissolved in 3 mol/hydrochloric acid/ethanol (10 ml) and heated until fully dissolved, and then the solution was cooled and precipitated to obtain 1.02 g of the compound V-2. This salt was shown to contain 0.5 equivalent of solvated water by elemental analysis.

MS(ESI): [M+H]⁺=460.

¹H-NMR(400 MHz,CDCl₃) δppm: 3.50(s, 6H), 3.85(m, 4H), 4.20(m, 2H), 4.28(m, 2H), 5.32(s, 2H), 7.05-7.10(m, 2H), 7.20(m,1H), 7.30(s,1H), 7.75(s,1H), 8.02(s,1H).

### EXAMPLE 3

### 9-fluoro-2,3-bis(2-methoxyethoxy)-8H-quinazolino[4,3-b]quinazoline mesylate (V-3)

4-chloro-6,7-bis(2-methoxyethoxy)quinazoline (3.12 g, 10 mmol) and 2-amino-6-fluoro-benzyl alcohol (1.41 g, 10 mmol) were dissolved in 20 ml isopropanol to form a solution, and 0.55 ml of concentrated hydrochloric acid was added dropwise to the solution. The reaction was carried out according to General Method Ito obtain a white solid intermediate M-3 (3.92 g, 85.96%).

M-3 (2.27 g, 5 mmol) was dissolved in 15 ml toluene, and cooled in an ice bath. Triethylamine (15 mmol) and methanesulfonyl chloride (20 mmol) were added to the reaction mixture dropwise sequentially. The reaction lasted for 7 h at room temperature. Water (30 ml) was added to the reaction mixture, and the reaction mixture was extracted by dichloromethane three times. The organic phase was combined, washed with water three times, then washed with saturated NaCl solution three times, and dried by anhydrous sodium sulfate. The solution was filtered to obtain a filtrate, which was concentrated under vacuum to obtain the crude product as a light yellow solid, which was recrystallized by ethanol or separated by column chromatography to obtain a white solid V-3′ (1.76 g, 88.22%).

The white solid V-3′ (2 mmol) obtained above was dissolved in hot ethanol, and methanesulfonic acid (2 mmol) was added dropwise into the ethanol solution. The solution was refluxed for 30 min, cooled and precipitated to obtain the compound V-3.

MS(ESI):[M+H]⁺=400.

¹H-NMR(400 MHz,CDCl₃) δppm: 3.44(s,6H), 3.85(m,4H), 4.26(m,2H), 4.41(m,2H), 5.61(s,2H), 7.12(s,1H), 7.18-7.30(m,3H), 7.6(8s,1H), 8.32(s, 1H).

### EXAMPLE 4

9-ethynyl-2,3-bis(2-methoxyethoxy)-8H-quinazolino[4,3-b]quinazoline hydrochloride trihydrate(V-4)

V-2 (5.07 g, 11 mmol), 2-methyl-3-butyn-2-ol (1.00 g, 10 mmol), palladium acetate (0.006 g, 0.025 mmol), copper iodide (0.012 g, 0.05 mmol) and triphenylphosphine (0.08 g, 0.3 mmol) were dissolved in 10 ml triethylamine, and refluxed for about 7 h under N₂. The reaction mixture was cooled to room temperature, and filtered to remove the insoluble substance. The filtrate was concentrated under the vacuum to obtain a brown oily substance. The oily substance was dissolved in ethyl acetate (30 ml), washed with aqueous solution of EDTA-2Na three times, with water three times, and with saturated NaCl solution three times. The organic layer was collected, and concentrated under vacuum to obtain a brown oil, which was recrystallized by isopropanol and dried to obtain a gray solid intermediate M-4 (3.34 g, 65.58%).

M-4 (1.00 g, 2 mmol) and NaOH (0.05 g, 1.25 mmol) were dissolved in toluene (30 ml), and heated to reflux for about 6 h under N₂. The solution was cooled to room temperature, and washed with water for 3 times. The organic layer was collected, and concentrated under vacuum to obtain a residue. The residue was subject to flash column chromatography, using a mobile phase of CH₂Cl₂ and EtOAc (1:10, VN) to obtain a gray solid V-4′ (0.35 g, 43.21%).

The gray solid V-4′ obtained above was dissolved in a hot solution of HCl in ethanol (3 mol/L, 10 ml), cooled and precipitated to obtain the compound V-4 (0.23 g). This V-4 was shown to contain 3 equivalents of solvated water by elemental analysis.

MS(ESI): [M+H]⁺=406.

¹H-NMR(400 MHz,CDCl₃) δppm: 3.50(s,6H), 3.80(m,4H), 4.12(m,2H), 4.26(m,2H), 4.26(m,2H), 4.56(s,2H), 5.36(s,2H), 7.05(s,1H), 7.24-7.38(m, 3H), 7.65(8s,1H), 8.62(s,1H).

### EXAMPLE 5

9-nitro-2,3-bis(2-methoxyethoxy)-8H-quinazolino[4,3-b]quinazoline (V-5)

4-chloro-6,7-bis(2-methoxyethoxy)quinazoline (3.12 g, 10 mmol) and 2-amino-6- nitro-benzyl alcohol (1.68 g, 10 mmol) (synthesized according to General Method III) were dissolved in 1,4-dioxane (20 ml), and concentrated hydrochloric acid (0.15 ml) was added. The reaction was allowed to react for 7 h at room temperature, and a white solid precipitated. The precipitate was filtered to obtain the crude product, which was recrystallized by ethanol and dried to obtain intermediate M-5 as a white solid (4.15 g, 86.46%).

M-5 (2.00 g, 4.16 mmol) was dissolved in CHCl₃ (15 ml), and in an ice water bath, triethylamine (0.5 ml) and methanesulfonyl chloride (0.4 ml) was added sequentially. The reaction was allowed to react for 7-10 h at room temperature. The reaction mixture was then added into water (30 ml) and extracted three times by CH₂Cl₂. The organic phase was combined, washed sequentially by water three times and saturated NaCl solution three times, and dried by anhydrous sodium sulfate. Then the organic phase was filtered to obtain a filtrate. The filtrate was concentrated under vacuum to obtain a crude compound, which was then recrystallized by alcohol or separated by column chromatography to obtain a white solid V-5 (1.21 g, 68.12%).

MS(ESI): [M+H]⁺=427.

¹H-NMR(400 MHz,CDCl₃) δppm: 3.50(s, 6H), 3.85(m, 4H), 4.30(m, 2H), 4.51(m, 2H), 5.42(s, 2H), 6.82(s, 1H), 7.19(m, 1H),7.29(s, 1H),7.38(m, 1H), 7.58(m, 1H), 7.65(s, 1H).

### EXAMPLE 6

### 9-amino-2,3-bis(2 -methoxyethoxy)-8H-quinazolino[4,3-b]quinazoline hydrobromide trihydrate(V-6)

V-5 (0.50 g, 1.17 mmol) and 10% Pd/C (0.10 g) were added in methanol (15 ml), heated to reflux, hydrogenated for 5 h under normal pressure, and then filtered. The filtrate was cooled in a freezer for 7 h, and a large amount of white substance precipitated. The precipitate was filtered and dried to obtain a white solid V-6′ (0.36 g, 77.70%).

The white solid V-6′ obtained above was dissolved in hot ethanol, and 2 ml of HBr/acetic ether solution (4 mol/L) was added. The solution was cooled and precipitated to obtain compound V-6, which was shown to contain 3 equivalents of solvated water by elemental analysis.

MS(ESI): [M+H]⁺=397.

¹H-NMR(400 MHz,DMSO-d6) δppm: 3.45(s, 6H), 3.85(m, 4H), 4.25(m, 2H), 4.40(m, 2H), 5.19(m, 2H), 6.39(s, J=6.4 Hz, 1H), 6.82-6.95(m, 2H), 7.00(s, 1H), 7.68(s, 1H), 8.00(s, 2H).

### EXAMPLE 7

### 9-cyano-2,3-bis(2-methoxyethoxy)-8H-quinazolino[4,3-b]quinazoline (V-7)

4-chloro-6,7-bis(2-methoxyethoxy)quinazoline (3.12 g,10 mmol) and 2-amino-6- cyano-benzyl alcohol (1.63 g, 11 mmol) were dissolved in isopropanol (20 ml), and diisopropylethylamine (0.50 ml) was added. The reaction was carried out according to General Method I for preparing the intermediate III to obtain a white solid intermediate M-6 (3.97 g, 86.30%).

M-6 (2.12 g, 5 mmol) was dissolved in CHCl₃ (15 ml) to form a solution, and in an ice water bath, triethylamine (15 mmol) and methanesulfonyl chloride (20 mmol) were added dropwise sequentially to the solution. The reaction was carried out according to General Method I for preparing compound V to obtain a white solid V-7(1.50 g, 59.17%).

MS(ESI): [M+H]⁺=508.

¹H-NMR(400 MHz,CDCl₃) δppm: 3.50(s, 6H), 3.82(m, 4H), 4.24(m, 2H), 4.33(m, 2H), 5.60(s, 2H), 7.20(s, 1H), 7.26-7.41(m, 3H), 7.89(s, 1H), 8.42(s, 1H).

### EXAMPLE 8

### 9-hydroxy-2,3-bis(2-methoxyethoxy)-8H-quinazolino[4,3-b]quinazoline (V-8)

4-chloro-6,7-bis(2-methoxyethoxy)quinazoline (3.12 g, 10 mmol) and 2-amino-6-hydroxyl-benzyl alcohol (1.39 g,10 mmol) were dissolved in isopropanol (20 ml), and concentrated hydrochloric acid (0.55 ml) was added dropwise. The reaction was carried out according to General Method I for preparing the intermediate III to obtain a white solid intermediate M-6 (3.55 g, 78.71%).

M-7(2.26 g, 5 mmol) was dissolved in CH₂Cl₂ (25 ml) to form a solution. In an ice water bath triethylamine (15 mmol) and methanesulfonyl chloride (20 mmol) were added dropwise sequentially to the solution. The reaction mixture was added into water (30 ml), and extracted by CH₂Cl₂ three times. The organic phase was combined, washed sequentially by water three times, and saturated NaCl solution three times, and then dried by anhydrous sodium sulfate. The organic phase was filtered to obtain a filtrate, which was concentrated under vacuum to obtain a light yellow compound intermediate M-8 (1.72 g, 62.89%).

M-8 (2.38 g, 5 mol) was dissolved in water (30 ml) to form a solution and 10% NaOH (2.0 ml) was added dropwise to the solution. The reaction mixture was refluxed for about 2 h, and then cooled to room temperature. The pH of the reaction mixture was adjusted to 5 by a hydrochloric acid (HCl) solution. A white solid precipitated. The precipitate was filtered to obtain compound V-8 (1.50 g, 75.57%).

MS(ESI): [M+H]⁺=398.

¹H-NMR(400 MHz,CDCl₃) δppm: 3.35(s, 6H), 3.85(m, 4H), 4.20(m, 2H), 4.40(m, 2H), 5.19(m, 2H), 5.51(s, 1H), 6.92(s,J=8.2 Hz, 1H), 7.02-7.24(m, 3H), 7.46(s, 1H), 8.05(s, 1H).

### EXAMPLE 9

9-trifluoromethyl-2,3-bis(2-methoxyethoxy)-8H-quinazolino[4,3-b]quinazoline (V-9)

4-chloro-6,7-bis(2-methoxyethoxy)quinazoline (3.12 g, 10 mmol) and 2-amino-6-trifluoromethyl-benzyl alcohol (2.10 g, 11 mmol) were dissolved in N,N-dimethylformamide (20 ml), and K₂CO₃ (4.14 g) was added. The reaction mixture was heated to 80° C., reacted for about 4 h, and cooled to room temperature. The reaction mixture was poured into cold water (100 ml), and a white solid precipitated. The precipitate was filtered to obtain a crude product. The crude product was recrystallized by 95% ethanol, and dried to obtain a white solid intermediate M-9 (4.17 g, 82.90%).

M-9 (2.52 g, 5 mmol) was dissolved in CHCl₃(15 ml), and in an ice water bath, triethylamine (15 mmol) and methanesulfonyl chloride (20 mmol) were added dropwise sequentially. The reaction was carried out according to General Method I for preparing the product V to obtain a white solid V-9 (1.66 g, 73.94%).

MS(ESI): [M+H]⁺=450.

¹H-NMR(400 MHz,CDCl₃) δppm: 3.45(s, 6H), 3.80(m, 4H), 4.35(m, 2H), 4.51(m, 2H), 5.32(s, 2H), 6.88(s, 1H), 7.05-7.22(m, 2H), 7.31(s, 1H), 7.44(m, 1H), 7.82(m, 1H), 8.05(s, 1H).

### EXAMPLE 10

### 9-methoxy-2,3-bis(2-methoxyethoxy)-8H-quinazolino[4,3-b]quinazoline (V-10)

4-chloro-6,7-bis(2-methoxyethoxy)quinazoline (3.12 g, 10 mmol) and 2-amino-6-methoxyl-benzyl alcohol (1.53 g, 10 mmol) were dissolved in acetonitrile (20 ml), and concentrated hydrochloric acid (0.55 ml) was added dropwise. The reaction was carried out according to General Method I for preparing intermediate III to obtain a white solid intermediate M-10 (4.01 g, 86.24%).

M-10 (2.33 g, 5 mmol) was dissolved in CH₂Cl₂ (15 ml). In an ice water bath triethylamine (15 mmol) and methanesulfonyl chloride (20 mmol) were added dropwise sequentially. The reaction was carried out according to General Method I for preparing the product V to obtain a white solid V-10 (1.23 g, 59.85%).

MS(ESI):[M+H]⁺=412.

¹H-NMR(400 MHz,CDCl₃) δppm: 3.50(s,6H), 3.79(s,3H), 3.84(m,4H), 4.32(m,2H), 4.56(m,2H), 5.28(s,2H), 6.72(s,1H), 7.01-7.18(m,2H), 7.31(s,1H), 7.41(m,1H), 7.66(m,1H), 7.90(s,1H).

### EXAMPLE 11

### N-{2,3-bis(2-methoxyethoxy)-8H-quinazolino[4,3-b]quinazoline}acetamide (V-11)

V-6 (0.50 g, 1.17 mmol) was dissolved in CHCl₃(10 ml), and in an ice water bath, acetyl chloride (0.05 mmol) was added dropwise. The reaction mixture was warmed to room temperature, reacted for about 3 h, and water (20 ml) was added. The reaction mixture was then extracted with CH₂Cl₂ three times. The organic phase was combined, washed sequentially with water three times and saturated NaCl solution three times, and dried by anhydrous sodium sulfate. The organic phase was then filtered to obtain a filtrate, which was concentrated under vacuum to obtain a crude product. The crude product was then recrystallized in alcohol and dried to obtain a white solid V-11 (0.41 g, 79.82%).

MS(ESI): [M+H]⁺=439.

¹H-NMR(400 MHz,CDCl₃) δppm: 2.32(s, 3H), 3.55(s, OCH₃, 6H), 3.84(m, 4H), 4.35(t, J=8.0 Hz, 2H), 4.49(t, J=8.2 Hz, 2H), 5.18(s, 2H), 6.72(s, 1H), 7.13-7.18(m, 2H), 7.31(s, 1H), 7.46(m, 1H), 7.66(m, 1H), 8.16(s, 1H), 9.01(s, NH, 1H).

### EXAMPLE 12

### N-{2,3-bis(2-methoxyethoxy)-8H-quinazolino[4,3-b]quinazoline}benzamide (V-12)

V-6 (0.50 g, 1.17 mmol) was dissolved in CHCl₃(10 ml). In an ice bath benzoyl chloride (0.18 g) was added dropwise. The reaction mixture was warmed to room temperature, and then reacted for about 3 h. The reaction mixture was then added into water (20 ml), and extracted with CH₂Cl₂ three times. The organic phase was combined, washed sequentially with water three times and saturated NaCl solution three times, and then dried by anhydrous sodium sulfate. The organic phase was filtered to obtain a filtrate. The filtrate was concentrated under vacuum to obtain a crude product, which was then recrystallized in alcohol and dried to obtain white solid V-12 (0.51 g, 87.18%).

MS (ESI): [M+H]⁺=501.

¹H-NMR(400 MHz, CDCl₃) δppm: 3.50(s, 2×CH₃, 6H), 3.84(m, 4H), 4.32(m, 4H), 5.24(s, 2H), 6.78(s, 1H), 6.85(m, 1H), 7.21-7.33(m, 3H), 7.56(m, 2H), 7.68(m, 1H), 7.80(m, 2H), 8.17(s, 1H), 9.15(s, NH, 1H).

### EXAMPLE 13

### 9-chloro-10-fluoro-2,3-bis(2-methoxyethoxy)-8H-quinazolino[4,3-b]quinazoline(V-13)

4-chloro-6,7-bis(2-methoxyethoxy)quinazoline (3.12 g, 10 mmol) and 2-chloro-3-fluoro-6-amino-benzyl alcohol (1.75 g, 10 mmol) were dissolved in N,N-dimethylacetamide (20 ml), and K₂CO₃ (4.10 g) was added. The reaction mixture was heated to 80° C., reacted for about 4 h, and then cooled to room temperature. The reaction mixture was then poured into cold water (100 ml), and a white solid precipitated. The precipitate-containing solution was filtered to obtain a crude product, which was recrystallized by 95% alcohol and dried to obtain a white solid intermediate M-11 (4.11 g, 84.39%).

M-11(2.44 g, 5 mmol) was dissolved in toluene (15 ml). In an ice water bath triethylamine (15 mmol) and methanesulfonyl chloride (25 mmol) were added dropwise sequentially. The reaction was carried out according to General Method I for preparing product V to obtain a white solid V-13 (1.55 g, 71.59%).

MS(ESI): [M+H]⁺=434.

¹H-NMR(400 MHz, CDCl₃) δppm: 3.50(s, 6H),3.85(m, 4H), 4.20(m, 2H), 4.28(m,2H), 5.32(s, 2H), 7.05-7.10(m, 2H), 7.20(m, 1H), 730(s, 1H), 7.75(s, 1H), 8.02(s, 1H).

### EXAMPLE 14

### 9-chloro-10-(3-fluoro-benzyloxy)-2,3-bis(2-methoxyethoxy)-8H-quinazolino[4,3-b]quinazoline (V-14)

4-chloro-6,7-bis(2-methoxyethoxy)quinazoline (3.12 g, 10 mmol) and (6-amino-2-chloro-3-((3-fluorophenyl oxy)phenyl)methanol (2.81 g, 10 mmol) were dissolved in isopropanol (30 ml), and HOAc (0.30 ml) was added. The reaction was carried out according to General Method I for preparing the product intermediate III to obtain a white solid intermediate M-12 (4.55 g, 76.72%).

M-12 (3.00 g, 5 mmol) was dissolved in CH₂Cl₂ (15 ml). In an ice water bath diisopropylethylamine (15 mmol) and methanesulfonyl chloride (25 mmol) were added dropwise sequentially. The reaction was carried out according to General Method I for preparing the product V to obtain a white solid V-14 (1.87 g, 69.39%).

MS (ESI): [M+H]⁺=540.

¹H-NMR(400 MHz,CDCl₃) δppm: 3.50(s, 6H),3.80(m, 4H), 4.25(m, 2H), 4.33(m, 2H), 5.26(s, 2H), 5.87(s, 2H),6.75-6.88(m, 2H), 6.95(m, 1H), 7.05(m, 1H), 7.20-7.29(m, 2H), 7.40(m, 1H), 7.78(s, 1H), 8.42(s, 1H).

### EXAMPLE 15

### 11-chloro-2,3-bis(2-methoxyethoxy)-8H-quinazolino[4,3-b]quinazoline (V-15)

4-chloro-6,7-bis(2-methoxyethoxy)quinazoline (3.12 g, 10 mmol) and 2-amino-4-chloro-benzyl alcohol (1.57 g, 10 mmol) were dissolved in isopropyl alcohol (30 ml), and concentrated hydrochloric acid (0.35 ml) was added. The reaction was carried out according to General Method I for preparing the intermediate III to obtain a white solid intermediate M-13 (4.05 g, 86.35%).

M-13 (2.35 g, 5 mmol) was dissolved in acetonitrile (15 ml) to form a solution, and in an ice water bath, pyridine (15 mmol) and methanesulfonyl chloride (20 mmol) were added dropwise into the solution sequentially. The reaction was carried out according to General Method I for preparing product V to obtain a white solid V-15 (1.32 g, 63.61%).

MS (ESI): [M+H]⁺=416.

¹H-NMR(400 MHz,CDCl₃) δppm: 3.45(s, 6H), 3.82(m, 4H), 4.25(t, J=8.4 Hz, 2H), 4.32(t, J=8.4 Hz, 2H), 5.46(s, 2H), 6.88(s, 1H), 7.01(m, 1H), 7.19(s,1H), 7.35-7.42(m, 2H), 8.66(s, 1H).

### EXAMPLE 16

### 11-fluoro-2,3-bis(2-methoxyethoxy)-8H-quinazolino[4,3-b]quinazoline (V-16)

4-chloro-6,7-bis(2-methoxyethoxy)quinazoline (3.12 g, 10 mmol) and 2-amino-4- fluoro-benzyl alcohol (1.41 g, 10 mmol) were dissolved in isopropyl alcohol (30 ml) to form a solution, and concentrated hydrochloric acid (0.55 ml) was added to the solution. The reaction was carried out according to General Method I for preparing the intermediate Ill to obtain a white solid intermediate M-14 (3.95 g, 87.20%).

M-14 (2.27 g, 5 mmol) and phosphorus oxychloride (3 ml) were reacted according to General Method I for preparing the product V to obtain a white solid V-16 (1.66 g, 83.21%).

MS (ESI): [M+H]⁺=400.

¹H-NMR(400 MHz,CDCl₃) δppm: 3.50(s, 6H), 3.80(m, 4H), 4.25(t, J=8.0 Hz, 2H), 4.30(t, J=8.0 Hz, 2H), 5.40(s, 2H), 6.76(s, 1H), 7.05-7.20(m, 3H), 7.46(s, 1H), 8.36(s, 1H).

### EXAMPLE 17

### 11-cyano-2,3-bis(2-methoxyethoxy)-8H-quinazolino[4,3-b]quinazoline (V-17)

4-chloro-6,7-bis(2-methoxyethoxy)quinazoline (3.12 g, 10 mmol) and 2-amino-4-cyano-benzyl alcohol (1.63 g, 11 mmol) were dissolved in isopropanol (20 ml) to form a solution, and HOAc (0.30 ml) was added to the solution. The reaction was carried out according to General Method I for preparing the intermediate III to obtain a white solid intermediate M-15 (3.72 g, 80.87%).

M-15 (2.12 g, 5 mmol) was dissolved in CHCl₃ (15 ml) to form a solution. In an ice water bath triethylamine (15 mmol) and methanesulfonyl chloride (20 mmol) were added dropwise into the solution sequentially. The reaction was carried out according to General Method I for preparing the product V to obtain a white solid V-17 (1.65 g, 65.08%).

MS (ESI): [M+H]⁺=407.

¹H-NMR(400 MHz,CDCl₃) δppm: 3.40(s, 6H), 3.75(m, 4H), 4.20(t, J=7.6 Hz, 2H), 4.28(t, J=8.0 Hz, 2H), 5.62(s, 2H), 6.82(s, 1H), 7.35-7.54(m, 3H), 7.66(s, 1H), 8.15(s, 1H).

### EXAMPLE 18

### 11-hydroxy-2,3-bis(2-methoxyethoxy)-8H-quinazolino[4,3-b]quinazoline (V-18)

According to the procedures of Example 8, 4-chloro-6,7-bis(2-methoxyethoxy)quinazoline (3.12 g,10 mmol) and 2-amino-4-hydroxyl-benzyl alcohol (1.39 g,10 mmol) were used to synthesize and obtain a white solid V-18 (2.03 g, the total yield was 51.13%).

MS (ESI): [M+H]⁺=398.

¹H-NMR(400 MHz,CDCl₃) δppm: 3.45(s, 6H), 3.75(m, 4H), 4.22(t, J=8.0 Hz, 2H), 4.28(t, J=8.0 Hz, 2H), 5.22(s, 2H), 5.46(s, 1H), 6.67(m, 1H), 6.78-6.90(m, 2H), 7.00(m, 1H), 7.25(s, 1H), 7.25(s, 1H), 8.15(s, 1H).

### EXAMPLE 19

11 -ethynyl-2,3-bis(2-methoxyethoxy)-8H-quinazolino[4,3-b]quinazoline (V-19)

According to the procedures of Example 4, V-15 (4.56 g, 11 mmol), 2-methyl-3-butyn-2-ol (1.00 g, 10 mmol), palladium acetate (0.006 g, 0.025 mmol), copper iodide (0.012 g, 0.05 mmol), triphenylphosphine (0.08 g, 0.3 mmol), and triethylamine (15 ml) were used to synthesize and obtain a white solid V-19.

MS (ESI): [M+H]⁺=406.

¹H-NMR(400 MHz, CDCl₃) δppm: 3.50(s, 6H), 3.64(m, 4H), 4.20(t, J=8.0 Hz, 2H), 4.32(t, J=8.0 Hz, 2H), 4.80(s, 1H), 5.62(s, 2H), 6.80(s, 1H), 7.26(m, 1H), 7.42-7.58(m, 3H), 8.31(s, 1H).

### EXAMPLE 20

### mesylate-{9-nitro-2,3-bis(2-methoxyethoxy)-8H-quinazolino[4,3-b]quinazoline-11-methanol}ester hydrochloride (V-20)

4-chloro-6,7-bis(2-methoxyethoxy)quinazoline (3.12 g, 10 mmol) and (2-amino-6-nitro-1,4-phenylene)dimethanol (2.18 g, 11 mmol) were dissolved in N,N-dimethylformamide (20 ml). The reaction was carried out according to General Method I for preparing the intermediate III to obtain a white solid intermediate M-16 (4.33 g, 84.90%).

M-16 (2.55 g, 5 mmol) was dissolved in toluene (25 ml) to form a solution. In an ice water bath triethylamine (15 mmol) and methanesulfonyl chloride (20 mmoI) were added dropwise into the solution sequentially. The reaction was carried out according to General Method I for preparing product V to obtain a white solid V-20′ (1.98 g, 74.16%).

The V-20 was prepared by converting the white solid V-20′ mentioned above according to the procedures of Example 1.

MS (ESI): [M+H]⁺=535.

¹H-NMR(400 MHz,CDCl₃) δppm: 3.15(s, 3H), 3.52(s, 6H), 3.85(m, 4H), 4.22-4.39(m, 4H), 5.25(s, 2H), 5.65(m, 2H), 7.12(s, 1H), 7.28(s, 1H), 7.70(s, 1H), 7.75(s, 1H), 7.82(s, 1H).

### EXAMPLE 21

9-amino-11-hydroxymethyl-2,3-bis(2-methoxyethoxy)-8H-quinazolino[4,3-b]quinazoline (V-21)

V-20 (1.00 g, 1.98 mmol) was dissolved in water (20 ml), and 10% KOH solution (1.5 ml) was added dropwise to the V-20 water solution. The reaction mixture was heated to reflux for 1 h, cooled to room temperature, and extracted by EtOAc three times. The organic phase was combined, washed with saturated NaCl solution for three times, and then dried by anhydrous sodium sulfate. The organic phase was filtered to obtain a filtrate. The filtrate was concentrated under vacuum to obtain a light yellow solid crude product. The crude product was recrystallized by petroleum ether-EtOAc (5:1, V/V) to obtain a gray solid intermediate M-17 (0.63 g, 69.62%).

M-17 (0.50 g, 1.10 mmol), 0.1 g 10%Pd/C and ethanol (10 ml) were heated to reflux, hydrogenated for 3 h under normal pressure. The reaction mixture was filtered to obtain a filtrate. The filtrate was concentrated under vacuum to obtain a brown oily product, which was recrystallized by ethanol to obtain a white solid V-21 (0.33 g, 70.42%).

MS (ESI): [M+H]⁺=427.

¹H-NMR (400 MHz,CDCl₃) δppm: 3.50(s, 6H), 3.80(m, 4H), 4.35(m, 4H), 4.58(s, 2H), 5.35(s, 2H), 6.55(s, 2H), 6.72(s, 1H), 7.25(s, 1H), 7.64(s, 2H), 8.26(s, 1H).

### EXAMPLE 22

### 11-chloro-8-methyl-2,3-bis(2-methoxyethoxy)-8H-quinazolino[4,3-b]quinazoline malate (V-22)

4-chloro-6,7-bis(2-methoxyethoxy)quinazoline (3.12 g, 10 mmol) and 1- (2-amino-4-chlorophenyl)ethanol (1.88 g, 11 mmol) were dissolved in N,N-dimethylacetamide (20 ml) to form a solution, and potassium carbonate (4.22 g) was added to the solution. The reaction mixture was heated to 80° C. and reacted for about 6 h, and cooled to room temperature. The reaction mixture was then poured into cold water (100 ml), and a white solid precipitated out. The precipitate was filtered to obtain a crude product, which was recrystallized with 95% ethanol and dried to obtain a white solid intermediate M-18 (4.03 g, 83A4%).

According to General Method I for preparing the product V, M-18 (1.00 g, 2 mmol) and phosphorus oxychloride (3 ml) were used to synthesize and obtain a white solid V-22′ (0.45 g, 52.45%).

The white solid V-22′ obtained above and malic acid were reacted according to the procedures of example 1 to obtain the V-22 (0.42 g).

MS (ESI): [M+H]⁺=430.

¹H-NMR(400 MHz,CDCl₃) δppm: 2.15(d,J=6.4 Hz,3H), 3.50(s,6H,2×OCH₃), 3.80(m,4H), 4.28(t,J=8.0 Hz,2H), 4.31(t,J=8.0 Hz,2H), 5.12(s,q,1H), 6.87(s, 1H), 7.11(s,1H), 7.25-7.32(m,2H), 7.46(s,1H), 8.35(s,1H).

### EXAMPLE 23

### 4-(3-((9-chloro-10-fluoro-3-methoxy-8H-quinazolino[4,3-b]quinazoline-2-yl)oxy)propyl)morpholine di-p-toluenesulfonate (V-23)

4-chloro-7-methoxy-6-(3-morpholino-4-yl-propoxy)quinazoline (3.37 g, 10 mmol) and 3-fluoro-2-chlorine-6-amino-benzyl alcohol (1.75 g, 10 mmol) were dissolved in isopropanol (20 ml) to form a solution, and concentrated hydrochloric acid (0.72 ml) was added dropwise into the solution. The reaction was carried out according to General Method I for preparing the intermediate Ill to obtain a white solid intermediate M-19 (4.03 g, 78.71%).

According to General Method I for preparing the product V, M-19 (1.00 g, 2 mmol) and phosphorus oxychloride (3 ml) were used to synthesize and obtain a white solid V-23′ (0.45 g, 52.45%).

The white solid V-23′ obtained above was reacted with p-toluene sulfonic acid to obtain the V-23. The V-23 was shown to contain two equivalents of toluene sulfonate and 3 equivalents of solvated water by elemental analysis.

MS (ESI): [M+H]⁺=459.

¹H-NMR(400 MHz,CDCl₃)δppm:2.15(m,2H),2.44-2.52(m,6H), 3.68(dd,J=8.4 Hz&8.0 Hz,4H), 3.80(s,3H), 4.17(t,J=7.6 Hz,2H), 5.26(s,2H), 7.02(s, 1H), 7.20(m, 1H), 7.26(s,1H), 7.34(m,1H), 7.86(s,1H), 836(s,1H).

### EXAMPLE 24

### 9-chloro-3-methoxy-2-(3-(pyrrolidine-1-yl)propoxy)-8H-quinazolino[4,3-b]quinazoline (V-24)

4-chloro-7-methoxy-6-(3-pyrrolidino-1-yl)propoxy)quinazoline (3.21 g, 10 mmol) and 2-chloro-6-amino-benzyl alcohol (1.57 g, 10 mmol) were dissolved in CH₂Cl₂ (65 ml) to form a solution, and diisopropyl ethylamine (0.35 ml) was added dropwise into the solution. The reaction was carried out according to General Method I for preparing the intermediate Ill to obtain a white solid intermediate M-20 (4.13 g, 86.40%).

According to General Method I for preparing product V, M-20 (1.00 g, 2 mmol) and phosphorus oxychloride (3 ml) were used to synthesize and obtain a white solid V-24 (0.40 g, 47.17%).

MS (ESI): [M+H]⁺=425.

¹H-NMR(400 MHz,DMSO-d6) δppm: 2.10(m,2H), 2.44-2.52(m,6H), 3.56(dd,J=8.4 Hz&8.6 Hz,4H), 3.80(s,3H), 4.22(t,J=7.8 Hz,2H), 5.30(s,2H), 6.90(s,1H), 7.20-7.35(m,3H), 7.55(s,1H), 8.26(s,1H).

### EXAMPLE 25

### 9-fluoro-3-methoxy-2-(3-(piperazinyl-1-yl)propoxy)-8H-quinazolino[4,3-b]quinazoline(V-25)

According to the procedures of Example 1, 4-chloro-7-methoxy-6-(3-(piperazinyl-1-yl)propoxy)quinazoline (3.36, 10 mmol) and 2-fluoro-6-amino benzyl alcohol (1.41 g, 10 mmol) were used to synthesize and obtain a white solid V-25 (2.00 g, 47.28%).

MS (ESI): [M+H]⁺=424.

¹H-NMR(400 MHz,DMSO-d6) δppm: 2.12(m,2H), 2.41-2.47(m,6H), 3.61(dd,J=8.0 Hz&8.2 Hz, 4H), 3.85(s, 3H), 4.41(t, J=8.0 Hz, 2H), 5.27(s, 2H), 6.95(s, 1H), 7.03-7.22(m,3H), 7.35 (s,1H), 8.14(s,1H).

### EXAMPLE 26

### 4-(3-((9-ethynyl-3-methoxy-8H-quinazolino[4,3-b]quinazoline-2-yl)oxy)propyl)morpholine (V-26)

4-chloro-7-methoxy-6-(3-(morpholino-4-yl-propoxy)quinazoline (3.37 g, 10 mmol) and 2-amino-6-acetylene benzyl alcohol (1.47 g, 10 mmol) were dissolved in isopropanol (20 ml) to form a solution, and concentrated hydrochloric acid (0.65 ml) was added dropwise into the solution. The reaction was carried out according to General Method I for preparing the intermediate Ill to obtain a white solid intermediate M-21 (4.03 g, 83.78%).

M-21 (2.42 g, 5 mmol) was dissolved in CH₂Cl₂ (25 ml) to form a solution, and in an ice water bath, triethylamine (15 mmol) and methanesulfonyl chloride (25 mmol) were added dropwise into the solution sequentially. The reaction was carried out according to General Method I for preparing the product V to obtain a white solid V-26 (1.38 g, 64.19%).

MS (ESI): [M+H]⁺=431.

¹H-NMR(400 MHz,DMSO-d6) δppm: 2.10(m,2H), 2.46-2.55(m,6H), 3.56(dd,J=8.4 Hz&8.0 Hz,4H), 3.72(s,3H), 4.20(s,1H), 4.32(t,J=8.0 Hz,2H), 5.25(s,2H), 6.95(s,1H),7.208-7.39(m,3H), 7.66 (s,1H), 8.45(s,1H).

### EXAMPLE 27

3-methoxy-2-(3-morpholinyl propoxy)-8H-quinazolino[4,3-b]quinazoline-9-nitrile(V-27)

4-chloro-7-methoxy-6-(3-(morpholino-4-yl-propoxy)quinazoline (3.37 g, 10 mmol) and 2-amino-6-nitrile benzyl alcohol (1.48 g, 10 mmol) were dissolved in isopropanol (20 ml) to form a solution, and formic acid (0.40 ml) was added dropwise into the solution. The reaction was carried out according to General Method I for preparing the intermediate III to obtain a white solid intermediate M-22 (4.00 g, 82.47%).

M-22 (2.43 g, 5 mmol) was dissolved in CH₂Cl₂ (25 ml) to form a solution, and in an ice water bath, triethylamine (15 mmol) and methanesulfonyl chloride (25 mmol) were added dropwise into the solution sequentially. According to General Method I for preparing the product V, the reaction was carried out to obtain a white solid V-27 (1.65 g, 76.57%).

MS (ESI): [M+H]⁺=432.

¹H-NMR(400 MHz,DMSO-d6) δppm: 2.10(m,2H), 2.46-2.55(m,6H), 3.60(dd,J=8.0 Hz&8.0 Hz,4H), 3.75(s,3H), 4.26(s,1H)4.35(m,2H), 5.45(s,2H), 7.05(s, 1H), 7.19(m,1H), 723-7.39(m,2H), 7.56 (s,1H), 8.35(s,1H).

### EXAMPLE 28

### 4-(3-((11-chloro-3-methoxy-8H-quinazolino[4,3-b]quinazoline-2-yl)oxy)propyl)morpholine (V-28)

4-chloro-7-methoxy-6-(3-(morpholino-4-yl-propoxy)quinazoline (3.37 g, 10 mmol) and 2-amino-4-chloro-benzyl alcohol (1.57 g, 10 mmol) were dissolved in isopropanol (20 ml), and concentrated hydrochloric acid (0.65 ml) was added dropwise into the reaction mixture. According to General Method I for preparing the intermediate III to obtain a white solid intermediate M-23 (3.90 g, 78.95%).

M-23 (1.48 g, 3 mmol) was dissolved in CH₂Cl₂ (25 ml), and in an ice water bath, triethylamine (10 mmol) and methanesulfonyl chloride (15 mmol) was added dropwise sequentially into the reaction mixture. The reaction was carried out according to General Method I for preparing the product V to obtain a white solid V-28 (0.85 g, 64.39%).

MS (ESI):[M+H]⁺=441.

¹H-NMR(400 MHz,DMSO-d6) δppm: 2.10(m,2H), 2.43(m,4H), 2.56(t,J=4.8 Hz), 3.53(dd,J=8.0 Hz&8.0 Hz,4H), 3.85(s,3H), 4.26(t,J=7.8 Hz,2H), 5.42(s,2H), 7.02(s,1H), 7.19(m,1H), 7.26(s,1H), 7.34-7.39(m,2H), 8.31(s,1H).

### EXAMPLE 29

### mesylate-{3-(((9-nitro--8H-quinazolino[4,3-b]quinazoline-2-yl)oxy)propyl)morpholine-11-methanol}ester (V-29)

4-chloro-7-methoxy-6-(3-(morpholino-4-yl-propoxy)quinazoline (3.37 g, 10 mmol) and 2-amino-6-nitro para-diphenyl methanol (1.48 g, 10 mmol) were dissolved in isopropanol (20 ml), and concentrated hydrochloric acid (0.55 ml) was added dropwise into the reaction mixture. The reaction was carried out according to General Method I for preparing the intermediate III to obtain a white solid intermediate M-24 (4.00 g, 82.47%).

M-24 (2.43 g, 5 mmol) was dissolved in CH₂Cl₂ (25 ml), and in an ice water bath triethylamine (15 mmol) and methanesulfonyl chloride (25 mmol) were added dropwise sequentially into the reaction mixture. The reaction was carried out according to General Method I for preparing the product V to obtain a white solid V-29 (1.65 g, 59.03%).

MS (ESI): [M+H]⁺=560.

¹H-NMR(400 MHz,CDCl₃) δppm: 2.10(m,2H), 2.45-2.56(m,6H), 3.30(s,3H), 3.50(dd,J=8.4 Hz&8.6 Hz,4H), 3.82(s,3H), 4.20(t,J=8.0 Hz,2H), 4.65(s,2H), 5.31(s,2H), 6.78(s,1H), 6.82(s,1H), 7.10(s,1H), 7.25(s,1H), 8.46(s,1H).

### EXAMPLE 30

### 9-chloro-10-fluoro-2,3-dimethoxy-8H-quinazolino[4,3-b]quinazoline (V-30)

4-chloro-6,7-bis(2-methoxyethoxy)quinazoline (2.24 g, 10 mol) and 2-chloro-3-fluoro-6-amino-benzyl alcohol (1.75 g, 10 mmol) were dissolved in N,N-dimethylformamide (15 ml), and K₂CO₃ (4.22 g) was added to the reaction mixture. The reaction mixture was heated to 80° C. and reacted for about 6 h, and cooled to room temperature. The reaction mixture was poured into cold water (100 ml), and a white solid precipitated. The precipitate was filtered to obtain a crude product, which was recrystallized with 95% ethanol and dried to obtain a white solid intermediate M-25 (2.87 g, 71.93%).

According to General Method I for preparing the product V, M-25 (1.20 g, 3 mmol) and phosphorus oxychloride (3 ml) were used to synthesize and obtain a white solid V-30 (0.62 g, 59.90%).

MS (ESI): [M+H]⁺=346.

¹H-NMR(400 MHz,CDCl₃) δppm: 3.70(s, 6H), 5.15(s, 2H), 6.95(s, 1H), 7.05-7.12(m, 2H), 7.55(s, 1H), 8.26(s, 1H).

### EXAMPLE 31

### 9-ethynyl-2,3-dimethoxy-8H-quinazolino[4,3-b]quinazoline

9-iodine-2,3-dimethoxy-8H-quinazoline[4,3-b]quinazoline (4.19 g, 10 mmol), 2-methyl-3-butyn-2-ol (1.00 g, 10 mmol), bis-triphenylphosphine palladium dichloride (0.018 g, 0.026 mmol) and copper iodide (0.012 g, 0.05 mmol) were dissolved in triethylamine (2 ml) and N,N-dimethylformamide (10 ml), and refluxed for about 6 h under N₂. The reaction mixture was cooled to room temperature, and filtered to remove insoluble substance. The filtrate was concentrated under vacuum to obtain a brown oily product. The oily product was dissolved by CH₂Cl₂ (30 ml), washed sequentially with aqueous solution of EDTA-2Na for three times, water three times, and saturated NaCl solution three times. The organic layer was collected and concentrated under vacuum to obtain a brown oily product. The brown oily product was recrystallized in isopropanol and dried to obtain a gray solid intermediate M-26 (2.76 g, 73.60%).

M-26 (1.00 g, 2.67 mmol) and NaOH (0.06 g, 1.5 mmol) were dissolved in toluene (30 ml) and water (5 ml), heated to reflux for about 6 h under N₂. The reaction mixture was cooled to room temperature, and washed with water three times. The organic layer was collected and concentrated under vacuum to obtain a residue. The residue was separated by flash column chromatography (washed with CH₂Cl₂-EtOAc solution, 1:10, V/V) to obtain a gray solid V-31 (0.71 g, 83.89%).

MS (ESI): [M+H]⁺=318.

¹H-NMR (400 MHz,DMSO-d₆) δppm: 3.82 (s,6H), 4.35 (s,1H), 5.06 (s,2H), 6.92 (s,1H), 7.20 (m,1H), 7.33 (s,1H), 7.44-7.56 (m,2H), 8.23 (s,1H).

### EXAMPLE 32

### 9-cyano-2,3-dimethoxy-8H-quinazolino[4,3-b]quinazoline (V-32)

4-chloro-6,7-bis(2-methoxyethoxy)quinazoline (2.24 g, 10 mol) and 2-amino-6-nitrile-benzyl alcohol (1.48 g, 10 mmol) were dissolved in isopropanol (20 ml), heated to reflux for about 6 h, filtered, and dried under vacuum to obtain a white solid intermediate M-27 (2.97 g, 79.84%).

According to General Method I for preparing the product V, M-27 (1.12 g, 3 mmol) and phosphorus oxychloride (2 ml) were used to synthesize and obtain a white solid V-32 (0.55 g, 57.65%).

MS (ESI): [M+H]⁺=319.

¹H-NMR (400 MHz,CDCl₃) δppm:3.72(s, 6H), 5.35(s, 2H),6.90(s, 1H), 7.25(m, 2H), 7.43(s, 1H), 7.50-7.62(m, 2H), 8.33(s, 1H).

### EXAMPLE 33

### N-((5-(9-chloro-10-(3-fluoro benzyloxy)-8H-quinazolino[4,3-b]quinazoline-2-yl)furan-2-yl)methyl)-2-(methylsulfonyl)ethylamine (V-33)

N-((5-(4-chloro-quinazoline-6-yl)furan-2-yl)methyl)-2-methylsulfonyl ethylamine (3.65 g, 10 mmol) and (6-amino-2-chloro-3-((3-fluoro-phenyl-oxy)phenyl)methanol (2.81 g, 10 mmol) were dissolved in isopropanol (30 ml), and concentrated hydrochloric acid (0.60 ml) was added dropwise. The reaction was carried out according to General Method I for preparing the intermediate III to obtain a white solid intermediate M-28 (4.75 g, 73.53%).

According to General Method I for preparing the product V, M-28 (2.28 g, 4 mmol) and phosphorus oxychloride (3 ml) were used to synthesize and obtain a white solid V-33 (1.35 g, 57.01%).

MS (ESI): [M+H]⁺=593.

¹H-NMR (400 MHz, DMSO-d₆) δppm: 3.00(t, 2H), 3.0 5(s, 3H), 3.36(t, 2H), 3.94(s, 2H), 4.05(s, NH, 1H), 5.26(s, 2H), 5.69(s, 2H), 6.80(d, J=8.8 Hz, 1H), 6.88-6.95(m, 2H), 7.00(s, 1H), 7.05(d, J=8.4 Hz, 1H), 7.18(m, 1H), 7.2 5˜7.35(m, 2H), 7.45 (m, 1H), 7.78(m, 1H), 8.16(s, 1H), 8.72(s, 1H).

### EXAMPLE 34

### N-((5-(9-chloro-10-fluoro-8H-quinazolino[4,3-b]quinazoline-2-yl)furan-2-yl)methyl)-2-(methylsulfonyl)ethylamine (V-34)

N-((5-(4-chloro-quinazoline-6-yl)furan-2-yl)methyl)-2-methylsulfonyl ethylamine (3.65 g, 10 mmol) and 2-chlorine-3-fluoro-6-amino-benzyl methanol (1.93 g, 11 mmol) were dissolved in acetonitrile (30 ml) to form a solution, and concentrated hydrochloric acid (0.55 ml) was added dropwise into the solution. The reaction was carried out according to General Method I for preparing the intermediate Ill to obtain a white solid intermediate M-29 (4.42 g, 81.85%).

According to General Method I for preparing the product V, M-29 (2.16 g, 4 mmol) and phosphorus oxychloride (5 ml) were used to synthesize and obtain a white solid V-34 (1.26 g, 64.81%).

MS (ESI): [M+H]⁺=487.

¹H-NMR (400 MHz, DMSO-d6) δppm: 3.00(t, 2H), 3.30(s, 3H), 3.45(t, 2H), 3.90(s, 2H), 4.15(s, NH, 1H), 5.56(s, 2H), 6.90(d, J=8.4 Hz, 1H), 7.00-7.25(m, 3H), 7.35(m, 1H), 7.48(m, 1H), 8.01(s, 1H), 8.3 5(s, 1H).

### EXAMPLE 35

### N-((5-(9-ethynyl-8H-quinazolino[4,3-b]quinazoline-2-yl)furan-2-yl)methyl)-2-(methylsulfonyl)ethylamine (V-35)

N-((5-(4-chloro-quinazoline-6-yl)furan-2-yl)methyl)-2-methylsulfonyl ethylamine (3.65 g, 10 mmol) and 2-amino-6-ethynyl benzyl alcohol (1.47 g, 10 mmol) were dissolved in acetonitrile (30 ml) to form a solution, and concentrated hydrochloric acid (0.60 ml) was added dropwise to the solution. The reaction was carried out according to General Method I for preparing the intermediate Ill to obtain a white solid intermediate M-30 (4.00 g, 78.13%).

M-30 (1.54 g, 3 mmol) was dissolved in CH₂Cl₂ (25 ml) to form a solution, and while in an ice water bath, triethylamine (9 mmol) and methanesulfonyl chloride (15 mmol) were added dropwise into the solution sequentially. The reaction was carried out according to General Method I for preparing the product V to obtain a white solid V-35 (0.65 g, 47.31%).

MS (ESI): [M+H]⁺=459.

¹H-NMR (400 MHz, DMSO-d6) δppm: 3.10(t, 2H), 3.25(s, 3H), 3.48(t, 2H), 3.90(s, 2H), 4.00(s, 1H), 4.25(s, NH, 1H), 5.35(s, 2H), 6.95(d, J=8.0 Hz, 1H), 7.06(m, 1H), 7.15-7.22(m, 3H), 7.28(m, 1H), 7.40(m, 1H), 7.81(s, 1H), 8.12(s, 1H).

### EXAMPLE 36

### N-((5-(11-chloro-8H-quinazolino[4,3-b]quinazoline-2-yl)furan-2-yl)methyl)-2-(methylsulfonyl)ethylamine (V-36)

N-((5-(4-chloro-quinazoline-6-yl)furan-2-yl)methyl)-2-methylsulfonyl ethylamine (3.65 g, 10 mmol) and 4-chlorine-2-amino-benzyl alcohol (1.73 g, 11 mmol) were dissolved in isopropanol (30 ml) to form a solution, and concentrated hydrochloric acid (0.60 ml) was added dropwise into the solution. The reaction was carried out according to General Method I for preparing the intermediate III to obtain a white solid intermediate M-31 (4.12 g, 76.58%).

According to General Method I for preparing the product V, M-31 (2.09 g, 4 mmol) and phosphorus oxychloride (5 ml) were used to synthesize and obtain a white solid V-36 (1.04 g, 55.55%).

MS (ESI): [M+H]⁺=469.

¹H-NMR (400 MHz, DMSO-d6) δppm: 3.05(t, 2H), 3.30(s, 3H), 3.45(t, 2H), 3.90(s, 2H), 4.06(s, NH, 1H), 5.15(s, 2H), 6.90(d, 1H), 7.01(m, 1H), 7.15(m, 1H), 7.23(s, 1H), 7.44(m, 1H), 7.58(m, 1H), 7.72(m, 1H), 7.96(s, 1H), 8.24(s, 1H).

### EXAMPLE 37

### 11-chloro-8-butyl-2,3-bis(2-methoxyethoxy)-8H-quinazolino[4,3-b]quinazoline(V-37)

4-chloro-6,7-bis(2-methoxyethoxy)quinazoline (3.12 g, 10 mmol) and 1-(2-amino-4-chlorophenyl)pentyl-1-alcohol (2.13 g, 10 mmol) were dissolved in isopropanol (30 ml) to form a solution, and concentrated sulfuric acid (0.20 ml) was added dropwise into the solution. The reaction was carried out according to General Method I for preparing the intermediate III to obtain a white solid intermediate M-32 (4.55 g, 86.67%).

M-32 (1.58 g, 3 mmol) was dissolved in CH₂Cl₂ (25 ml) to form a solution. In an ice water bath triethylamine (9 mmol) and methanesulfonyl chloride (15 mmol) were added sequentially into the solution. The reaction was carried out according to General Method I for preparing the product V to obtain a white solid V-37 (0.97 g, 68.65%).

MS (ESI): [M+H]⁺=472.

¹H-NMR(400 MHz, DMSO-d6)δppm:1.25(t, J=7.8 Hz, 3H), 1.60-1.65(m, 4H), 2.12(q, J=7.6 Hz, 2H), 318(5, 6H), 3.96(m, 4H), 4.20(t, J=4.8 Hz, 2H), 4.3(t, J=4.4 Hz, 2H), 4.92(t, J=4.8 Hz, 1H), 6.85(s, 1H), 7.17(m, 1H), 7.40(m, 2H), 7.44(s, 1H), 8.25(s, 1H).

### EXAMPLE 38

### 11-chloro-2,3-bis(2-methoxyethoxy)-8-(2-methoxyethyl)-8H-quinazolino[4,3-b]quinazoline(V-38)

4-chloro-6,7-bis(2-methoxyethoxy)quinazoline (3.12 g, 10 mmol) and 1-(2-amino-4-chlorophenyl)ethyl-1-alcohol (1.71 g, 10 mmol) were dissolved in isopropanol (30 ml) to form a solution, and concentrated hydrochloric acid (0.60 ml) was added dropwise to the solution. The reaction was carried out according to General Method I for preparing the intermediate III to obtain a white solid intermediate M-33 (3.25 g, 67.29%).

M-33 (1.45 g, 3 mmol) was dissolved in CH₂Cl₂ (25 ml) to form a solution, and in an ice water bath triethylamine (9 mmol) and methanesulfonyl chloride (15 mmol) were added dropwise sequentially to the solution. The reaction was carried out according to General Method I for preparing the product V to obtain a white solid V-38 (1.03 g, 72.59%).

MS (ESI): [M+H]⁺=474.

¹H-NMR(400 MHz, DMSO-d6) δppm: 3.05(q, 2H), 3.80(s, 9H), 3.92(t, J=4.4 Hz, 2H), 4.20(t, J=4.8 Hz, 4H), 4.35(t, J=4.4 Hz, 2H), 4.46(t, J=4.4 Hz, 2H), 5.21(t, J=4.8 Hz, 1H), 6.90(s, 1H), 7.06(m, 1H), 7.22(m, 1H), 7.40(m, 2H), 7.45(m, 1H), 8.33(s, 1H).

### EXAMPLE 39

### 2-chloro-11-cyano-8H-quinazolino[4,3-b]quinazoline (V-39)

According to the procedures of Example 1, 4,6-dichloro-quinazoline (1.98 g, 10 mmol) and 2-amino-4-cyano-benzyl alcohol (1.48 g, 10 mmol) were used to synthesize and obtain a white solid V-39 (1.54 g, the total yield of two steps was 52.74%).

MS (ESI): [M+H]⁺=293.

¹H-NMR (400 MHz, DMSO-d6) δppm: 4.40(s, 2H), 7.22(m, 1H), 7.40(m, 1H), 7.50(m, 1H), 7.52-7.55(m, 2H), 8.00(s, 1H), 8.22(s, 1H).

### EXAMPLE 40

### 11-chloro-2-nitro-8H-quinazolino[4,3-b]quinazoline (V-40)

According to the procedures of Example 1, 4-chloro-6-nitro-quinazoline (1.57 g, 10 mmol) and 2-amino-4-chloro-benzyl alcohol (2.09 g, 10 mmol) were used to synthesize and obtain a yellow solid V-40 (1.79 g, the total yield of the two steps was 57.37%).

MS (ESI): [M+H]⁺=313.

¹H-NMR (400 MHz, DMSO-d6) δppm: 4.15(s, 2H), 7.10(m, 1H), 7.26(s, 1H), 7.40(m, 1H), 7.52(m, 1H), 8.10(s, 1H), 8.22(m, 1H), 8.65(s, 1H).

### EXAMPLE 41

11-chloro-2-amino-8H-quinazolino[4,3-b]quinazoline (V-41)

V-40 (0.32 g, 1 mmol) was dissolved in ethanol (10 ml), and stannous chloride dihydrate (0.67 g, 3 mmol) was added to form a yellow and clear solution. The reaction lasted for 7 h at room temperature. The reaction mixture was concentrated under vacuum to obtain a residue. The residue was added into about 20 ml of water, and the pH was adjusted to 8 with 10% sodium hydroxide solution. A large quantity of solid then precipitated. The precipitate was filtered to obtain a crude product, which was then recrystallized with ethanol to obtain a white solid V-41 (2.20 g, 78.01%).

MS (ESI): [M+H]⁺=283.

¹H-NMR (400 MHz, DMSO-d6) δppm: 4.26(s, 2H), 6.25(s, NH₂, 2H), 6.95(m, 1H), 7.05(s, 1H), 7.10(m, 1H), 7.18 (m,1H), 7.28(s, 1H), 7.50(m, 1H), 8.15(s, 1H).

### EXAMPLE 42

### 11-chloro-2-methoxy-3-trifluoromethyl-8H-quinazolino[4,3-b]quinazoline(V-42)

4-chloro-6-methoxy-7-trifluoromethyl quinazoline (2.62, 10 mmol) and 2-amino-4-chloro-benzyl alcohol (1.57 g, 10 mol) were dissolved in isopropanol (30 ml) to form a solution, and concentrated hydrochloric acid (0.55 ml) was added dropwise to the solution. The reaction was carried out according to General Method I for preparing the intermediate III to obtain a white solid intermediate M-34 (3.33 g, 79.47%).

M-34 (1.26 g, 3 mmol) was dissolved in CH₂Cl₂ (25 ml) to form a solution, and in an ice water bath triethylamine (9 mmol) and methanesulfonyl chloride (15 mmol) were added dropwise sequentially to the solution. The reaction was carried out according to General Method I for preparing the product V to obtain a white solid V-42 (0.84 g, 7611%).

MS (ESI): [M+H]⁺=366

¹H-NMR (400 MHz, CDCl₃) δppm: 3.80(s, 3H), 4.65(s, 2H), 7.10(d, J=8.0 Hz, 1H), 726(5, 1H), 7.40(m,1H), 7.58(s, 1H), 7.62(s, 1H), 8.09(s, 1H).

### EXAMPLE 43

### 11-chloro-2-(3-fluoro-benzyloxy)3-methoxy-8H-quinazolino[4,3-b]quinazoline (V-43)

4-chloro-2- (3-fluoro-benzyloxy)-7-methoxy-quinazoline (3.18, 10 mmol) and 4-chloro-2-amino-benzyl alcohol (1.57 g, 10 mmol) were dissolved in isopropanol (30 ml) to form a solution, and phosphoric acid (0.20 ml) was added dropwise to the solution. The reaction was carried out according to General Method I for preparing the intermediate III to obtain a white solid intermediate M-35 (3.83 g, 80.63%).

M-35 (1.43 g, 3 mmol) was dissolved in CH₂Cl₂ (25 ml) to form a solution, and in an ice water bath triethylamine (9 mmol) and methanesulfonyl chloride (15 mmol) were added dropwise sequentially to the solution. The reaction was carried out according to General Method I for preparing the product V to obtain a white solid V-43 (0.72 g, 57.01%).

MS (ESI): [M+H]⁺=422.

¹H-NMR (400 MHz, DMSO-d6) δppm: 3.80(s, 3H), 4.25(s, 2H), 5.36(s, 2H), 6.85(s, 1H), 6.97(s, 1H), 7.17-7.30(m, 4H), 7.40-7.42(m, 2H), 7.55(s, 1H), 8.18(s, 1H).

### EXAMPLE 44

### 9-bromo-3-methoxy-2-n-propyl-8H-quinazolino[4,3-b]quinazoline (V-44)

4-chloro-6-n-propyl-7-methoxy-quinazoline (2.36, 10 mmol) and 6-bromo-2-amino-benzyl alcohol (2.01 g, 10 mmol) were dissolved in isopropanol (35 ml), and heated to reflux for about 5 h. The reaction was carried out according to General Method I for preparing the intermediate III to obtain a white solid intermediate M-36 (3.65 g, 83.52%).

M-36 (1.75 g, 4 mmol) was dissolved in CH₂Cl₂ (25 ml) to form a solution, and in an ice water bath triethylamine (12 mmol) and methanesulfonyl chloride (20 mmol) were added dropwise sequentially to the solution. The reaction was carried out according to General Method I for preparing the product V to obtain a white solid V-44 (0.98 g, 63.97%).

MS (ESI): [M+H]⁺=384.

¹H-NMR (400 MHz, DMSO-d6) δppm: 1.52(t, J=4.4 Hz, 3H), 1.98(m, 2H), 3.05(t, J=4.8 Hz, 2H), 3.85(s, 3H), 4.46(s, 2H), 6.95(d, J=8.0 Hz, 1H), 7.08(m, 1H), 7.22(m, 1H), 7.40(m, 1H), 7.58(s, 1H), 8.34(s, 1H).

### EXAMPLE 45

### 9-bromo-8H-quinazolino[4,3-b]quinazoline-2- formic acid ethyl ester (V-45)

4-chloro-quinazoline-6-formic ether (2.36, 10 mmol) and 6-bromo-2-amino-benzyl alcohol (2.01 g, 10 mmol) were dissolved in isopropanol (30 ml) to form a solution, and concentrated hydrochloric acid (0.55 ml) was added dropwise to the solution. The reaction lasted for 7 h at room temperature, and the reaction mixture was filtered to obtain a white solid, which was dried under vacuum to obtain intermediate M-37 (3.32 g, 75.97%).

M-37 (1.31 g, 3 mmol) was dissolved in CH₂Cl₂ (25 ml) to form a solution, and in an ice water bath triethylamine (9 mmol) and methanesulfonyl chloride (12 mmol) were added dropwise sequentially to the solution. The reaction was carried out according to General Method I for preparing the product V to obtain a white solid V-45 (0.82 g, 71.18%).

MS (ESI): [M+H]⁺=384.

¹H-NMR (400 MHz, DMSO-d6) δppm: 2.10(t, J=8.0 Hz, 3H), 4.00(q, J=7.6 Hz, 2H), 5.15(s, 2H), 7.06(m, 1H), 7.15 (m, 1H), 7.48-7.52(m, 2H), 8.25(m, 1H), 8.32(s, 1H), 8.66(s, 1H).

### EXAMPLE 46

### (E)-(9-bromo-8H-quinazolino[4,3-b]quinazoline-2-yl)-4(dimethylamino)-2-butene amide hydrochloride (V-46)

According to the procedures of Example 5, N-(4-chloro-quinazoline-6-yl)-3-(dimethylamino) acrylamide (2.76 g, 10 mmol) and 4-bromo-2-amino-benzyl alcohol (2.20 g, 11 mmol) were used to obtain V-46 (1.26 g), an off white solid compound V-46′ (the total yield of two steps was 29.79%).

The solid compound V-46′ obtained above was converted to V-46 according the procedures disclosed herein.

MS (ESI): [M+H]⁺=438.

¹H-NMR (400 MHz, DMSO-d6) δppm: 3.25(s, 6H), 3.42(d, J=4.6 Hz, 2H), 4.20(s, 2H), 5.35(d, J=16.2 Hz, 1H), 6.87(m, 1H), 7.12(m, 1H), 7.30(s, 1H), 7.51-7.55(m, 1H), 2H), 7.81(s, 1H), 8.23(s, 1H), 8.76(d, J=11.2 Hz, 1H), 10.24(s, 1H, NH).

### EXAMPLE 47

### 9-bromo-8H-quinazolino[4,3-b]quinazoline-2-formic acid (V-47)

V-45 (0.25 g, 0.65 mmol) was dissolved in ethanol (15 ml) to form a solution, and 10% NaOH solution (0.15 ml) was added dropwise to the solution. The reaction lasted for 6 h at room temperature. Then the reaction mixture was dried under vacuum to obtain a residue. The residue was added to 10m1 of water, and the pH was adjusted to 5 with 10% hydrochloric acid solution. A white solid precipitated. The precipitate was filtered to obtain a white solid V-47 (0.21 g,91.01%).

MS (ESI): [M+H]⁺=356

¹H-NMR (400 MHz, DMSO-d6) δppm: 4.20(s, 2H),6.88(m, 1H), 7.12(d, J=8 Hz, 1H), 7.22(m, 1H), 7.42(m, J=8.2 Hz, 1H), 7.65(m, 1H), 8.23(d, J=8.4 Hz, 1H), 8.56(s, 1H), 8.90(s, 1H), 11.21(s, 1H, OH).

### EXAMPLE 48

### 4-(2,3-dimethoxy-8H-quinazolino[4,3-b]quinazoline-9-yl)morpholine (V-48)

4-chloro-6,7-bis(2-methoxyethoxy)quinazoline (0.23 g, 1 mmol) and 1-(2-amino-6-morpholinyl-phenyl)-methanol (0.21 g, 1 mmol) were dissolved in CHCl₃ (50 ml) to form a solution, and concentrated hydrochloric acid (0.90 ml) was added to the solution. The reaction was carried out according to General Method I for preparing the intermediate Ill to obtain a white solid intermediate M-38 (0.32 g, 72.73%).

M-38 (0.43 g, 1 mmol) was dissolved in CH₂Cl₂ (7 ml) to form a solution, and in an ice water bath, diisopropylethylamine (3 mmol) and methanesulfonyl chloride (5 mmol) the added dropwise sequentially to the solution. The reaction was carried out according to General Method I for preparing the product V to obtain a white solid V-48 (0.12 g, 3175%).

MS (ESI): [M+H]⁺=379

¹H-NMR (400 MHz, DMSO-d6) δppm: 3.25(m, 4H), 3.50(m, 4H), 3.85(s, 6H), 5.20(s, 2H), 6.50(d, J=8.0 Hz, 1H), 6.72(d, J=8.2Hz, 1H), 6.95(s, 1H), 7.12(m, 1H), 7.48(s, 1H), 8.43(s, 1H).

### EXAMPLE 49

### 11-chloro-2,3-dimethoxy-8-trifluoromethyl-8H-quinazolino[4,3-b]quinazoline (V-49)

Concentrated hydrochloric acid (0.55 ml) was added dropwise to 6,7-bis(2-methoxyethoxy)quinazoline (0.23 g, 1 mmol) and 1-(2-amino-4- Chlorophenyl)-2,2,2-trifluoro-benzyl alcohol (0.23 g, 1 mmol). The reaction was carried out according to General Method I for preparing the intermediate Ill to obtain a white solid intermediate M-39 (0.35 g, 76.09%).

M-39 (0.46 g, 1 mmol) was dissolved in CH₂Cl₂ (7 ml) to form a solution, and while the solution was in an ice water bath triethylamine (3 mmol) and methanesulfonyl chloride (5 mmol) were added dropwise sequentially to the solution. The reaction was carried out according to General Method I for preparing the product V to obtain a white solid V-49 (0.33 g, 83.54%).

MS (ESI): [M+H]⁺=396.

¹H-NMR (400 MHz, DMSO-d6) δppm: 3.85(s, 6H), 5.44(s, 2H), 6.87(s, 1H), 7.20(m, 1H), 7.33(s, 1H), 7.41(m, 1H), 7.46(s, 1H), 8.76(s, 1H).

### EXAMPLE 50

### 4-chloro-6,7-bis(2-methoxyethoxy)quinazoline (compound I-1)

Ethyl 3,4-dihydroxybenzoate (36.4 g, 0.2 mol), potassium carbonate (82.8 g, 0.6 mol), and 2-methoxyethyl p-toluenesulfonate (72.0 g, 0.4 mol) were dissolved in acetonitrile (200 ml), heated and refluxed for 6 h. The solvent of the reaction mixture was evaporated under vacuum to obtain a residue. The residue was recrystallized with isopropanol, suction filtered, and dried under vacuum to obtain a white powder 3,4-bis (2-methoxyethoxy)ethyl benzoate (53.2 g, 89%).

3,4-bis(2-methoxyethoxy)benzoic acid ethyl ester (15.00 g, 0.05 mol) was added into acetic acid (50 ml) to form a mixture, and while the mixture was stirred in an ice water bath, 65-68% nitric acid (13 ml) was added to the mixture. The mixture then was stirred for 24 h at room temperature. The reaction mixture was transferred into ice water (500 ml), and extracted with ethyl acetate. The organic phase was combined, washed with saturated sodium bicarbonate solution three times, washed with saturated sodium chloride solution, and then dried by anhydrous sodium sulfate. The organic phase was then filtered to obtain a filtrate. The filtrate was concentrated to obtain a brown oily liquid 2-nitro-4,5-bis(2-methoxyethoxy)ethyl benzoate (14.10 g, 82.22%).

2-nitro-4,5-bis(2-methoxyethoxy)benzoic acid ethyl ester (34.3 g, 0.10 mol), ammonium formate (63 g, 1.00 mol), 5% Pd/C (5.00 g), and formamide (150 ml) were reacted for 7 h at 150° C. The reaction mixture was cooled to room temperature, and a solid precipitated. The precipitate was filtered to obtain a white powder 6,7-bis-(2-methoxyethoxy)-4(3H)quinazolinone (22.1 g, 75%).

Thionyl chloride (14.9 g) was added dropwise slowly to a solution of 6,7-bis-(2-methoxyethoxy)-4 (3H) quinazolinone (14.7 g, 0.15 mol), N,N-dimethylformamide (1 ml), and dichloromethane (150 ml). The reaction mixture was stirred at room temperature, and refluxed for 6 h. The reaction mixture was cooled to room temperature, and the pH of the reaction mixture was adjusted to 7-8 with a sodium hydroxide solution. The reaction mixture was then stirred for 30 min at room temperature, and set aside to allow the organic and aqueous layers to separate. The organic layer was collected, and concentrated under vacuum to obtain a white solid 4-chloro-6,7-bis-(2-methoxyethoxy)-quinazoline (14.1 g, 89.5%), [M₊H]⁺=313.

### EXAMPLE 51

### 2-amino-6-bromobenzyl alcohol (intermediate I-1)

2-nitro-6-bromo-toluene (32.20 g, 0.20 mol), N-bromosuccinimide (26.6 g, 0.20 mol), and benzoyl peroxide (0.40 g, 2 mmol) were dissolved in chlorobenzene (100 ml), and heated to reflux for about 20 h. The reaction was stopped, and the reaction mixture was filtered to obtain a filtrate. The filtrate was concentrated under vacuum to obtain a light brown oily product, which was recrystallized with ethanol to obtain a yellow solid 2-nitro-6-bromo benzyl bromide (47.31 g, 81%).

2-nitro-6-bromo benzyl bromide (14.70 g, 0.05 mol) and anhydrous sodium acetate(14.7 g,0.15 mol) were added into N,N-dimethylformamide (70 ml), heated and reacted for about 1 h at 70° C., and cooled to room temperature. The reaction mixture was added into water (150 ml), extracted with ethyl acetate three times, washed with water three times, washed with saturated sodium chloride solution three times, dried by anhydrous sodium sulfate, and filtered. The filtrate was concentrated under vacuum to obtain a white solid 2-nitro-6-bromo phenyl acetic acid methyl ester (12.65 g, 92%).

2-nitro-6-bromo phenyl acetic acid methyl ester (2.73 g, 0.01 mol) was added into the water (100 ml), and 10% potassium hydroxide solution (20 ml) was added dropwise. The reaction mixture was heated to reflux for 2 h, cooled to room temperature, and extracted with ethyl acetate for three times. The organic phase was combined, washed with water and with saturated sodium chloride solution sequentially, dried by anhydrous sodium sulfate, and filtered. The filtrate was concentrated under vacuum to obtain a yellow solid 2-bromo-6-nitro-benzyl alcohol (1.91 g, 83%).

2-bromo-6-nitro methanol (1.15 g, 5 mmol), sodium sulfide (0.78 g, 10 mmol), and sulfur (0.32 g, 10 mmol) were dissolved in ethanol (50 ml) and water (25 ml), heated to reflux for 2 h, and then cooled to room temperature. The ethanol was evaporated by concentration under vacuum, and the rest of the reaction mixture was added into water (30 ml), and extracted with ether three times. The organic phase was combined, washed with water and with saturated sodium chloride solution sequentially, dried by anhydrous sodium sulfate, and filtered. The filtrate was concentrated under vacuum to obtain a yellow solid 2-bromo-6-amino-benzyl alcohol (0.78 g, 78%), [M+H]⁺=202.

### EXAMPLE 52

The inhibitory activities of EGFR tyrosine kinase in vitro

The inhibitory activity of compounds V-1 to V-36 on EGFR tyrosine kinase in vitro was tested according to the instruction of an EGFR inhibitor screening kit (Cat #PV3193, Invitrogen). The 200 μM and 40 μM solutions of compounds V-1 to V-36 were prepared, and the enzyme inhibition activities of the compounds V-1 to V-36 were tested at these two concentrations. Erlotinib hydrochloride was used as a positive control drug. The test results are listed in Table 1.

TABLE 1
The inhibition ratio of the compound on EGFR(%)

| | The inhibition ratio of the | |
|---|---|---|
| | compound on EGFR(%) | |
| Number | 200 μM | 40 μM |
| V-1 | 91.5 | 57.6 |
| V-2 | 97.9 | 62.9 |
| V-3 | 81.6 | 49.5 |
| V-4 | 93.2 | 48.7 |
| V-5 | 74.6 | 25.2 |
| V-6 | 93.8 | 55.2 |
| V-7 | 80.5 | 36.2 |
| V-8 | 95.6 | 52.3 |
| V-9 | 60.3 | 13.0 |
| V-10 | 78.5 | 30.8 |
| V-11 | 85.6 | 56.4 |
| V-12 | 62.7 | 25.8 |
| V-13 | 88.9 | 45.7 |
| V-14 | 87.9 | 45.2 |
| V-15 | 94.0 | 60.7 |
| V-16 | 80.1 | 21.7 |
| V-17 | 70.6 | 25.4 |
| V-18 | 78.8 | 33.2 |
| V-19 | 95.7 | 53.4 |
| V-20 | 98.4 | 59.9 |
| V-22 | 95.6 | 63.2 |
| V-24 | 94.3 | 53.1 |
| V-28 | 98.7 | 64.4 |
| V-29 | 86.5 | 35.2 |
| V-30 | 82.1 | 27.8 |
| V-31 | 76.5 | 30.6 |
| V-33 | 97.9 | 64.3 |
| V-34 | 72.6 | 21.6 |
| V-35 | 75.8 | 13.3 |
| V-36 | 87.9 | 55.6 |
| Erlotinib hydrochloride | 81.7 | 37.9 |

### EXAMPLE 53

The inhibitory activities of aurora kinase B in vitro

The inhibitory activities of compounds V-1 to V-36 on aurora kinase in vitro were tested according to the instructions of an aurora kinase screening kit (Cat #PV3174, Invitrogen). The 200 μM and 40 μM solutions of compounds V-1 to V-36 were prepared, and the enzyme inhibition activities of compounds V-1 to V-36 were tested at these concentrations. Compound VX-680 (N-(4-(4-((5-methyl-1-H-pyrazole-3-yl)amino)-6-(4-methyl piperazine-1-yl)pyrimidine-2-yl)-thio)phenyl)cyclopropane carboxamide, an aurora kinase inhibitor) was used as a positive control drug. The testing results are listed in Table 2.

TABLE 2
The inhibition ratio of the compound on Aurora B(%)

| | The inhibition ratio of the | |
|---|---|---|
| | compound on Aurora B(%) | |
| Number | 200 μM | 40 μM |
| V-1 | 88.6 | 47.6 |
| V-2 | 95.8 | 53.2 |
| V-3 | 83.6 | 39.5 |
| V-4 | 91.5 | 38.7 |
| V-5 | 74.6 | 35.1 |
| V-6 | 93.8 | 48.5 |
| V-7 | 91.7 | 23.2 |
| V-8 | 96.4 | 32.3 |
| V-9 | 60.5 | 17.6 |
| V-10 | 73.6 | 20.8 |
| V-11 | 96.7 | 53.5 |
| V-12 | 87.9 | 40.1 |
| V-13 | 95.8 | 55.2 |
| V-14 | 76.9 | 23.2 |
| V-15 | 92.5 | 58.7 |
| V-16 | 78.1 | 21.5 |
| V-17 | 84.3 | 26.4 |
| V-18 | 76.9 | 23.2 |
| V-19 | 95.7 | 52.4 |
| V-20 | 96.4 | 58.9 |
| V-22 | 95.6 | 57.2 |
| V-24 | 90.3 | 52.5 |
| V-28 | 78.7 | 45.2 |
| V-29 | 78.9 | 11.2 |
| V-30 | 79.6 | 46.3 |
| V-31 | 66.5 | 10.6 |
| V-33 | 93.7 | 54.6 |
| V-34 | 92.6 | 17.8 |
| V-35 | 79.8 | 3.3 |
| V-36 | 97.9 | 52.6 |
| VX-680 | 81.6 | 35.2 |

The structure of VX-680 is shown below:

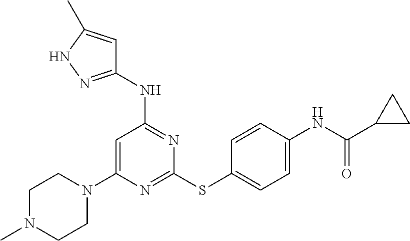

### EXAMPLE 54

The inhibitory activities of the compounds on tumor cells in vitro

The inhibition ratios of the compounds on Hut78 T-Lymphocytic Leukemia cells, Jurkat E6-1 human T-cell lymphoma, PANC-1 human pancreatic cancer cells, A549 human lung cancer cells, K562 human chronicity marrow leukemic cells, Hep3B2.1-7 human hepatoma carcinoma cells, MDA-MB-435s human breast carcinoma cells, Colo320 human rectal cancer cells, PC-3 human carcinoma of prostate, and HepG2 human hepatoma carcinoma cells were determined using a CCK-8 kit. The concentrations of the compounds tested were 100 μM and 10 μM. Erlotinib hydrochloride and gefitinib were used as positive control drugs. The test results are listed in Table 3 and Table 4.

TABLE 3
Antiproliferative effect of compounds on Jurkat E6-1, Hut78,
Colo320, K562, and 435s cells

| | E6-1 | | Hut-78 | | Colo320 | | K562 | | 435s | |
|---|---|---|---|---|---|---|---|---|---|---|
| | 100 | 10 | 100 | 10 | 100 | 10 | 100 | 10 | 100 | 10 |
| Number | μM | μM | μM | μM | μM | μM | μM | μM | μM | μM |
| V-1 | 81.67 | 55.01 | 64.54 | 16.15 | 80.98 | 55.14 | 87.03 | 40.66 | 91.20 | 60.85 |
| V-2 | 83.12 | 56.24 | 73.79 | 28.95 | 89.60 | 56.99 | 91.10 | 65.44 | 82.59 | 59.83 |
| V-3 | 78.02 | 42.02 | 84.14 | 44.14 | 78.98 | 34.77 | 88.79 | 43.14 | 84.12 | 3.38 |
| V-4 | 19.79 | 6.85 | 85.01 | 44.10 | 83.38 | 48.91 | 87.58 | 31.44 | 92.57 | 31.31 |
| V-5 | 78.86 | 34.78 | 63.55 | 43.77 | 84.94 | 51.70 | 78.27 | 23.63 | 87.20 | 48.94 |
| V-6 | 95.35 | 61.09 | 91.87 | 68.97 | 99.15 | 73.46 | 82.83 | 64.13 | 86.97 | 50.42 |
| V-7 | 75.38 | 34.56 | 62.41 | 32.06 | 81.38 | 42.11 | 61.68 | 24.29 | 81.39 | 37.96 |
| V-8 | 85.41 | 44.00 | 75.93 | 35.58 | 94.85 | 64.49 | 92.19 | 52.43 | 93.71 | 55.02 |
| V-9 | 65.43 | 20.40 | 70.55 | 19.24 | 73.29 | 46.27 | 79.88 | 34.63 | 80.74 | 54.85 |
| V-10 | 84.15 | 51.83 | 72.25 | 41.08 | 72.81 | 10.76 | 84.99 | 47.54 | 65.10 | 29.59 |
| V-11 | 90.80 | 41.62 | 72.78 | 30.99 | 96.30 | 41.56 | 85.98 | 50.45 | 89.51 | 41.01 |
| V-13 | 97.97 | 52.59 | 101.39 | 66.16 | 483.64 | 59.70 | 98.46 | 66.15 | 67.92 | 26.76 |
| V-14 | 97.71 | 26.85 | 98.69 | 21.28 | 88.00 | 17.56 | 100.49 | 36.61 | 94.77 | 46.62 |
| V-15 | 85.81 | 69.49 | 93.20 | 60.98 | 96.46 | 58.29 | 91.51 | 45.20 | 92.03 | 72.61 |
| V-16 | 97.30 | 56.63 | 99.57 | 44.27 | 99.40 | 52.71 | 93.75 | 53.63 | 66.20 | 48.94 |
| V-17 | 79.56 | 33.69 | 45.28 | 35.91 | 26.05 | 9.66 | 25.20 | 19.15 | 95.49 | 37.48 |
| V-18 | 84.05 | 44.09 | 65.15 | 19.72 | 75.06 | 38.13 | 59.03 | 10.45 | 72.26 | 36.58 |
| V-19 | 91.12 | 55.24 | 63.79 | 32.95 | 89.60 | 36.99 | 81.10 | 45.44 | 91.59 | 55.83 |
| V-20 | 95.63 | 57.91 | 82.14 | 52.33 | 92.27 | 66.86 | 97.44 | 40.66 | 93.88 | 65.72 |
| V-21 | 90.31 | 43.50 | 52.69 | 10.33 | 87.01 | 31.75 | 81.57 | 26.92 | 74.86 | 66.97 |
| V-22 | 98.02 | 67.02 | 94.64 | 54.14 | 98.98 | 43.77 | 88.79 | 23.14 | 94.12 | 63.38 |
| V-23 | 77.52 | 56.28 | 99.80 | 42.03 | 99.54 | 52.32 | 73.37 | 58.67 | 52.17 | 20.68 |
| V-24 | 87.30 | 46.63 | 99.57 | 44.27 | 99.40 | 52.71 | 93.75 | 53.63 | 66.20 | 48.94 |
| V-25 | 81.35 | 31.09 | 21.87 | 18.97 | 79.15 | 33.46 | 82.83 | 44.13 | 76.97 | 20.42 |
| V-26 | 85.38 | 44.56 | 72.41 | 32.06 | 81.38 | 32.11 | 11.68 | 4.29 | 71.39 | 37.96 |
| V-27 | 75.41 | 24.00 | 95.93 | 65.58 | 84.85 | 34.49 | 62.19 | 22.43 | 79.71 | 12.02 |
| V-28 | 95.43 | 40.40 | 90.55 | 59.24 | 63.29 | 16.27 | 99.88 | 64.63 | 90.74 | 54.85 |
| V-31 | 84.15 | 21.83 | 72.25 | 41.08 | 92.81 | 30.76 | 84.99 | 37.54 | 85.10 | 50.59 |
| V-33 | 90.80 | 41.62 | 92.78 | 60.99 | 96.30 | 61.56 | 85.98 | 40.45 | 99.51 | 62.74 |
| Erlotinib | 75.63 | 47.91 | 62.14 | 42.33 | 62.27 | 56.86 | 67.44 | 40.66 | 83.88 | 35.72 |
| Hydrochloride | | | | | | | | | | |
| Gefitinib | 70.12 | 40.59 | 70.36 | 35.41 | 65.42 | 44.31 | 78.52 | 10.02 | 80.01 | 31.13 |

TABLE 4
Antiproliferative effect of compounds on Hep3B, A549, PANC-1,
PC-3 cells

| | Hep3B | | A549 | | PANC-1 | | PC-3 | |
|---|---|---|---|---|---|---|---|---|
| | 100 | 10 | 100 | 10 | 100 | 10 | 100 | 10 |
| Number | μM | μM | μM | μM | μM | μM | μM | μM |
| V-1 | 89.26 | 50.37 | 81.43 | 40.52 | 78.10 | 35.67 | 62.37 | 51.62 |
| V-2 | 90.00 | 53.45 | 90.69 | 68.62 | 95.91 | 59.74 | 92.73 | 72.25 |
| V-3 | 80.66 | 42.32 | 76.22 | 44.59 | 75.40 | 30.87 | 86.95 | 33.98 |
| V-4 | 79.00 | 33.45 | 78.69 | 48.62 | 85.91 | 39.74 | 62.73 | 22.25 |
| V-5 | 54.40 | 22.27 | 92.45 | 59.76 | 80.30 | 17.08 | 63.57 | 32.50 |
| V-6 | 84.67 | 51.34 | 97.30 | 63.01 | 98.57 | 64.17 | 84.37 | 53.36 |
| V-7 | 89.48 | 40.43 | 83.61 | 27.69 | 86.58 | 40.71 | 90.30 | 15.86 |
| V-8 | 87.27 | 52.44 | 79.97 | 52.80 | 89.78 | 41.46 | 93.96 | 45.18 |
| V-9 | 82.77 | 40.14 | 87.51 | 33.72 | 55.56 | 11.72 | 78.03 | 16.37 |
| V-10 | 72.21 | 31.90 | 82.78 | 28.77 | 96.90 | 36.52 | 91.73 | 9.39 |
| V-11 | 82.82 | 57.10 | 99.18 | 63.07 | 91.27 | 57.93 | 89.67 | 55.07 |
| V-13 | 102.34 | 64.71 | 91.08 | 43.04 | 87.24 | 45.92 | 87.74 | 61.35 |
| V-14 | 100.18 | 45.14 | 97.84 | 31.10 | 99.90 | 38.29 | 97.08 | 38.52 |
| V-15 | 97.36 | 52.36 | 90.76 | 75.64 | 94.14 | 61.77 | 81.32 | 42.36 |
| V-16 | 74.56 | 30.33 | 70.22 | 50.69 | 99.90 | 22.38 | 98.89 | 53.50 |
| V-17 | 85.22 | 58.35 | 87.14 | 49.89 | 56.76 | 19.56 | 91.67 | 28.12 |
| V-18 | 77.45 | 40.15 | 78.56 | 37.79 | 84.38 | 30.01 | 76.16 | 36.99 |
| V-19 | 85.00 | 53.45 | 90.69 | 48.62 | 85.91 | 49.74 | 92.73 | 62.25 |
| V-20 | 92.23 | 60.73 | 90.70 | 55.03 | 88.25 | 54.79 | 75.15 | 46.75 |
| V-21 | 86.31 | 21.82 | 78.04 | 45.28 | 89.44 | 52.67 | 62.58 | 27.61 |
| V-22 | 90.66 | 62.32 | 96.22 | 54.59 | 95.40 | 60.87 | 96.95 | 63.98 |
| V-23 | 48.66 | 27.12 | 22.52 | 10.43 | 98.80 | 21.54 | 97.60 | 30.69 |
| V-24 | 94.56 | 60.33 | 98.22 | 50.69 | 99.90 | 42.38 | 98.89 | 53.50 |
| V-25 | 64.67 | 21.34 | 79.30 | 47.01 | 88.57 | 44.17 | 64.37 | 53.36 |
| V-26 | 89.48 | 28.43 | 93.61 | 27.69 | 76.58 | 40.71 | 30.30 | 5.86 |
| V-27 | 67.27 | 2.44 | 79.97 | 39.80 | 99.78 | 41.76 | 83.96 | 25.18 |
| V-28 | 92.77 | 40.14 | 97.51 | 53.72 | 95.56 | 51.72 | 88.03 | 56.37 |
| V-31 | 62.21 | 31.90 | 32.78 | 28.77 | 96.40 | 46.52 | 81.73 | 29.39 |
| V-33 | 86.48 | 51.30 | 86.06 | 48.09 | 87.99 | 50.30 | 78.15 | 16.47 |
| Erlotinib | 52.23 | 30.73 | 85.70 | 34.03 | 78.25 | 14.79 | 54.15 | 24.75 |
| Hydrochloride | | | | | | | | |
| Gefitinib | 70.31 | 25.52 | 76.82 | 30.01 | 58.83 | 21.37 | 55.46 | 26.72 |

### EXAMPLE 55

The inhibitory activities of the compounds on tumor cells (IC₅₀) in vitro

The IC₅₀ (IC₅₀, unit: μg/ml) values of the compounds on Hut78 T-Lymphocytic Leukemia cells, Jurkat E6-1 human T-cell lymphoma, PANC-1 human pancreatic cancer cells, A549 human lung cancer cells, K562 human chronicity marrow leukemic cells, Hep3B2. 1-7 human hepatoma carcinoma cells, MDA-MB-435s human breast carcinoma cells, Colo320 human rectal cancer cells, PC-3 human carcinoma of prostate, and HepG2 human hepatoma carcinoma cells were determined using a CCK-8 kit. Erlotinib hydrochloride was used as the positive control drug. The test results are listed in Table 5.

TABLE 5
IC₅₀ of compounds on E6-1, Hut-78, Colo320, K562, 435s,
Hep3B, A549, PANC-1, and PC-3 cells

| Number | E6-1 | Hut-78 | Colo320 | K562 | 435s | Hep3B | A549 | PANC-1 | PC-3 |
|---|---|---|---|---|---|---|---|---|---|
| V-2 | 15.31 | 61.15 | 32.31 | 33.96 | 0.42 | 9.83 | 0.67 | 0.11 | 4.78 |
| V-6 | 30.46 | 58.75 | 29.78 | 46.35 | 33.46 | 15.60 | 3.68 | 16.78 | 32.99 |
| V-8 | 8.11 | 53.12 | 29.76 | 63.12 | 27.63 | 33.15 | 30.01 | 21.17 | 9.90 |
| V-11 | 39.88 | 26.78 | 34.56 | 40.59 | 6.79 | 26.11 | 4.64 | 6.72 | 36.79 |
| V-13 | 3.89 | 0.62 | 9.88 | 10.99 | 31.43 | 0.32 | 10.72 | 14.88 | 9.78 |
| V-15 | 5.72 | 14.33 | 55.62 | 1.50 | 21.00 | 6.91 | 1.01 | 8.32 | 24.62 |
| V-19 | 12.46 | 47.38 | 10.03 | 31.11 | 36.54 | 9.32 | 50.03 | 27.34 | 15.33 |
| V-20 | 0.05 | 55.31 | 13.14 | 10.10 | 16.78 | 71.43 | 12.13 | 3.72 | 22.66 |
| V-22 | 13.33 | 11.36 | 4.67 | 19.64 | 6.18 | 14.41 | 9.87 | 84.31 | 7.69 |
| V-24 | 9.86 | 2.14 | 4.76 | 10.32 | 53.36 | 43.12 | 23.27 | 1.94 | 3.41 |
| V-28 | 10.01 | 16.71 | 59.68 | 7.84 | 16.31 | 36.75 | 6.42 | 18.77 | 31.43 |
| V-33 | 15.39 | 13.42 | 7.83 | 62.56 | 10.30 | 22.70 | 0.05 | 1.97 | 40.66 |
| Erlotinib | 35.64 | 43.21 | 40.39 | 39.88 | 13.12 | 62.31 | 19.33 | 32.54 | 54.21 |
| Hydrochloride | | | | | | | | | |

### EXAMPLE 56

### Acute Toxicity Study

The acute toxicity experiments were carried out according to the methods disclosed in the book of Modern Pharmacological Methods, edited chiefly by Zhang Jun-Tian. Through preliminary screening using the Bliss statistical method, the LD₅₀ of compounds V-2, V-15, V-20, V-22, and V-33 were determined to be 2.05 g/kg, 2.97 g/kg, 0.75 g/kg, 1.34 g/kg, and 2.84 g/kg, respectively.

### EXAMPLE 57

Tablets of the compounds disclosed herein can be made according to the following recipe:

| | |
|---|---|
| At least one active pharmaceutical | 10 mg |
| ingredient disclosed herein | |
| sucrose | 150 mg |
| corn starch | 38 mg |
| calcium striethylaminerate | 2 mg |

To a mixture of at least one active pharmaceutical ingredient, cane sugar and corn starch is added an appropriate amount of water. The mixture is stirred until it is uniform. The mixture is then dried, crushed, and sieved. Calcium striethylaminerate is then added to the mixture and mixed well, and then the mixture is pressed into tablets. Each tablet weighs 200mg, which contains 10mg pharmaceutically active ingredient.

### EXAMPLE 58

A formulation for injection can be made according to the following recipe:

| | |
|---|---|
| At least one active pharmaceutical | 20 mg |
| ingredient disclosed herein | |
| Water for injection | 80 mg |
| Preparation method | |

The at least one active pharmaceutical ingredient can be dissolved in water for injection, mixed, and filtered. The solution obtained can be subpackaged into sterile ampoule bottles under sterile conditions. The solution in one bottle can be 10 mg, which contains 2mg of the at least one active pharmaceutical ingredient disclosed herein.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. At least one active pharmaceutical ingredient chosen from polycyclic quinazolines of formula V, 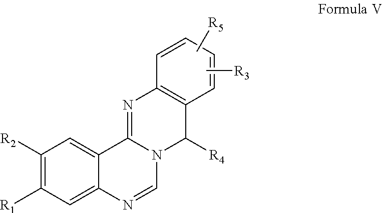
pharmaceutically acceptable salts thereof, and hydrates of the pharmaceutically acceptable salts,
wherein, R₁ and R₂ are independently chosen from hydrogen, halogen, substituted and unsubstituted amino, nitro, carboxyl, substituted and unsubstituted C₁-C₄ alkoxy carbonyl, substituted and unsubstituted C₁-C₄ alkyl, substituted and unsubstituted alkoxy, guanidino, ureido, substituted and unsubstituted C₁-C₄ sulfonyl, substituted and unsubstituted aryl sulfonyl, substituted and unsubstituted aryl, substituted and unsubstituted heterocyclic, and substituted and unsubstituted heteroaryl;
R₃ and R₅ are independently chosen from hydrogen, halogen, cyano, nitro, substituted and unsubstituted amino, hydroxyl, substituted and unsubstituted C₁-C₄ alkyl, substituted and unsubstituted C₁-C₄ alkoxy, substituted and unsubstituted C₂-C₄ unsaturated aliphatic chain, substituted and unsubstituted five-membered aliphatic ring, substituted and unsubstituted six-membered aliphatic ring, substituted and unsubstituted aryl, substituted and unsubstituted heterocyclic, and substituted and unsubstituted heteroaryl;
R₄ is chosen from hydrogen, substituted and unsubstituted C₁-C₄ alkyl, and substituted and unsubstituted C₁-C₄ alkoxy; and
R₁, R₂, R₃, R₄ and R₅ are not simultaneously hydrogen.

2. The at least one active pharmaceutical ingredient according to claim 1, wherein R₁, R₂, R₃, and/or R₅ are independently chosen from substituted and unsubstituted phenyl.

3. The at least one active pharmaceutical ingredient according to claim 2, wherein the substituted phenyl has 1 to 4 substituents independently chosen from halogen, hydroxyl, nitro, cyano, substituted and unsubstituted C₁-C₄ alkoxy, substituted and unsubstituted C₁-C₄ alkyl, and NH₂.

4. The at least one active pharmaceutical ingredient according to claim 1, wherein R₁, R₂, R₃, and/or R₅ are independently chosen from substituted and unsubstituted benzyloxy.

5. The at least one active pharmaceutical ingredient according to claim 4, wherein R₁, R₂, R₃, and/or R₅ are independently chosen from 3-F-benzyloxy.

6. The at least one active pharmaceutical ingredient according to claim 1, wherein R₁, R₂, R₃ and/or R₅ are independently chosen from (1) saturated and partially unsaturated, substituted and unsubstituted five-membered heterocyclic; and (2) saturated and partially unsaturated, substituted and unsubstituted six-membered heterocyclic; wherein the substituted and unsubstituted heterocyclic contains at least one hetero atom independently chosen from nitrogen, oxygen, and sulfur.

7. The at least one active pharmaceutical ingredient according to claim 1, wherein R₁ and/or R₂ are independently chosen from hydroxyl amino, substituted and unsubstituted aryl acyl amino, C₁-C₄ acyl amino, and (E)-4-(dimethylamino)-2-butene acylamino.

8. The at least one active pharmaceutical ingredient according to claim 1, wherein R₃ and/or R₅ are independently chosen from C₁-C₄ acyl amino, benzoyl-amino, and C₁-C₄ alkyl amino.

9. The at least one active pharmaceutical ingredient according to claim 1, wherein R₃ and/or R₅ are independently chosen from trifluoromethyl, hydroxymethyl, and alkyl sulfonate.

10. The at least one active pharmaceutical ingredient according to claim 1, wherein R₁, R₂, and/or R₄ are independently chosen from trifluoromethyl.

11. The at least one active pharmaceutical ingredient according to claim 1, wherein R₁, R₂, R₃, and/or R₅ are independently chosen from 5-(((2-(methylsulfonyl)ethyl)amino)methyl)furyl.

12. The at least one active pharmaceutical ingredient according to claim 1, wherein R₁ and/or R₂ are independently chosen from C₁-C₄ alkoxy, benzyloxy, pyrrolidine-1-yl-(C₂-C₄)alkoxy, morpholino-1-yl-(C₂-C₄)alkoxy, piperazin-l-yl-(C₂-C₄)alkoxy, N-methyl piperazine-1-yl-(C₂-C₄) alkoxy, and piperidine-1-yl-(C₂-C₄)alkoxy.

13. The at least one active pharmaceutical ingredient according to claim 1, wherein R₁, R₂, R₃, and/or R₅ are independently chosen from methoxy, 3-F-benzyloxy, pyrrolidine-1-yl-2- propoxy, morpholino-1-yl-2- propoxy, piperazin-1-yl-2-propoxy, N-methyl piperazine-1-yl-2- propoxy, and piperidine-1-yl-2- propoxy.

14. The at least one active pharmaceutical ingredient according to claim 1, wherein the halogen in R₁, R₂, R₃, and/or R₅ is independently chosen from fluorine, chlorine, and bromine.

15. The at least one active pharmaceutical ingredient according to claim 1, wherein the pharmaceutically acceptable salts are chosen from hydrochloride, hydrobromide, sulfate, acetate, lactate, tartrate, tannate, citrate, triflouroacetate, malate, maleate, succinate, p-toluenesulfonate, and mesylate.

16. The at least one active pharmaceutical ingredient according to claim 1, wherein the hydrates of the pharmaceutically acceptable salts have a hydrate number chosen from 0.5, 1, 2, and 3.

17. The at least one active pharmaceutical ingredient according to claim 1, wherein R₁, R₂, R₃, R₄, and/or R₅ are independently chosen from methoxy, ethoxy, n-propoxy, isopropoxy, n-butoxy, isobutoxy, methoxyethyl, 2-methoxy ethoxy, and ethoxy methoxy.

18. The at least one active pharmaceutical ingredient according to claim 1, wherein R₁ and/or R₂ are independently chosen from alkoxy, NH₂, nitro, trifluoromethyl, C₁-C₄ alkyl, C₁-C₄ alkoxy carbonyl, carboxyl, and (E)-4-(dimethylamino)-2-butene acylamino.

19. The at least one active pharmaceutical ingredient according to claim 1, wherein R₁ and/or R₂ are independently chosen from hydrogen, chlorine, n-propyl, NH₂, nitro, trifluoromethyl, methoxy, 2-methoxy-ethoxy, 3-fluoro-benzyloxy, pyrrolidine-1-yl-2- propoxy, morpholino-1-yl-2- propoxy, piperazin-1-yl-2- propoxy, N-methyl piperazine-1-yl-2- propoxy, piperidine-1-yl-2- propoxy, ethoxycarbonyl, carboxyl, 5-(((2-(methylsulfonyl)ethyl)amino)methyl)furyl, and (E)-4-(dimethylamino)-2-butene acylamino.

20. The at least one active pharmaceutical ingredient according to claim 1, wherein R₃ and/or R₅ are independently chosen from hydrogen, halogen, cyano, nitro, NH₂, unsaturated aliphatic chain, alkoxy, hydroxyl, trifluoromethyl, alkyl sulfonate, hydroxymethyl, heterocyclic and C₁-C₄ acyl amino.

21. The at least one active pharmaceutical ingredient according to claim 1, wherein R₃ and/or R₅ are independently chosen from substituted and unsubstituted C₂-C₄ alkynyl.

22. The at least one active pharmaceutical ingredient according to claim 1, wherein R₃ and/or R₅ are independently chosen from hydrogen, fluorine, chlorine, bromine, cyano, nitro, NH₂, hydroxyl, trifluoromethyl, acetylene, MeS(O)₂O—CH₂—, MeC(O)NH—, PhC(O)NH—, methoxy, hydroxymethyl, morpholino and 3-fluoro-benzyloxy.

23. The at least one active pharmaceutical ingredient according to claim 1, wherein R₄ is chosen from hydrogen, methyl, methoxyethyl, trifluoromethyl, and n-butyl.

24. At least one polycyclic quinazoline chosen from:
9-chloro-2,3-bis(2-methoxyethoxy)-8H-quinazolino[4,3-b]quinazoline,
9-bromo-2,3-bis(2-methoxyethoxy)-8H-quinazolino[4,3-b]quinazoline,
9-fluoro-2,3-bis(2-methoxyethoxy)-8H-quinazolino[4,3-b]quinazoline,
9-ethynyl-2,3-bis(2-methoxyethoxy)-8H-quinazolino[4,3-b]quinazoline,
9-nitro-2,3-bis(2-methoxyethoxy)-8H-quinazolino[4,3-b]quinazoline,
9-amino-2,3-bis(2-methoxyethoxy)-8H-quinazolino[4,3-b]quinazoline,
9-cyano-2,3-bis(2-methoxyethoxy)-8H-quinazolino[4,3-b]quinazoline,
9-hydroxy-2,3-bis(2-methoxyethoxy)-8H-quinazolino[4,3-b]quinazoline,
9-trifluoromethyl-2,3-bis(2-methoxyethoxy)-8H-quinazolino[4,3-b]quinazoline,
9-methoxy-2,3-bis(2-methoxyethoxy)-8H-quinazolino[4,3-b]quinazoline,
N-{2,3-bis(2-methoxyethoxy)-8H-quinazolino[4,3-b]quinazoline}acetamide,
N-{2,3-bis(2-methoxyethoxy)-8H-quinazolino[4,3-b]quinazoline}benzamide,
9-chloro-10-fluoro-2,3-bis(2-methoxyethoxy)-8H-quinazolino[4,3-b]quinazoline,
9-chloro-10-(3-fluoro-benzyloxy)-2,3-bis(2-methoxyethoxy)-8H-quinazolino[4,3-b]quinazoline,
11-chloro-2,3-bis(2-methoxyethoxy)-8H-quinazolino[4,3-b]quinazoline,
11-fluoro-2,3-bis(2-methoxyethoxy)-8H-quinazolino[4,3-b]quinazoline,
11-cyano-2,3-bis(2-methoxyethoxy)-8H-quinazolino[4,3-b]quinazoline,
11-hydroxy-2,3-bis(2-methoxyethoxy)-8H-quinazolino[4,3-b]quinazoline,
11-ethynyl-2,3-bis(2-methoxyethoxy)-8H-quinazolino[4,3-b]quinazoline, mesylate-{9-nitro-2,3-bis(2-methoxyethoxy)-8H-quinazolino[4,3-b]quinazoline-11-methanol}ester,
9-amino-11-hydroxymethyl-2,3-bis(2-methoxyethoxy)-8H-quinazolino[4,3-b]quinazoline,
11-chloro-8-methyl-2,3-bis(2-methoxyethoxy)-8H-quinazolino[4,3-b]quinazoline,
4-(3-((9-chloro-10-fluoro-3-methoxy-8H-quinazolino[4,3-b]quinazoline-2-yl)oxy)propyl)morpholine,
9-chloro-3-methoxy-2-(3-(pyrrolidine-1-yl)propoxy)-8H-quinazolino[4,3-b]quinazoline,
9-fluoro-3-methoxy-2-(3-(piperazinyl-1-yl)propoxy)-8H-quinazolino[4,3-b]quinazoline,
4-(3-((9-ethynyl-3-methoxy-8H-quinazolino[4,3-b]quinazoline -2-yl)oxy)propyl)morpholine,
3-methoxy-2-(3-morpholinyl propoxy)-8H-quinazolino[4,3-b]quinazoline-9-nitrile,
4-(3-((11-chloro-3-methoxy-8H-quinazolino[4,3-b]quinazoline-2-yl)oxy)propyl)morpholine,
mesylate-{3-(((9-nitro--8H-quinazolino[4,3-b]quinazoline-2-yl)oxy)propyl)morpholine-11-methanol}ester,
9-chloro-10-fluoro-2,3-dimethoxy-8H-quinazolino[4,3-b]quinazoline,
9-ethynyl-2,3-dimethoxy-8H-quinazolino[4,3-b]quinazoline,
9-cyano-2,3-dimethoxy-8H-quinazolino[4,3-b]quinazoline,
N-((5-(9-chloro-10-(3-fluoro benzyloxy)-8H-quinazolino[4,3-b]quinazoline-2-yl)furan-2-yl)methyl)-2-(methylsulfonyl)ethylamine,
N-((5-(9-chloro-10-fluoro-8H-quinazolino[4,3-b]quinazoline-2-yl)furan-2-yl)methyl)-2-(methylsulfonyl)ethylamine,
N-((5-(9-ethynyl-8H-quinazolino[4,3-b]quinazoline-2-yl)furan-2-yl)methyl)-2-(methylsulfonyl)ethylamine,
N-((5-(11-chloro-8H-quinazolino[4,3-b]quinazoline-2-yl)furan-2-yl)methyl)-2-(methylsulfonyl)ethylamine,
11-chloro-8-n-butyl-2,3-bis(2-methoxyethoxy)-8H-quinazolino[4,3-b]quinazoline,
11-chloro-2,3-bis(2-methoxyethoxy)-8-(2-methoxyethyl)-8H-quinazolino[4,3-b]quinazoline,
2-chloro-11-cyano-8H-quinazolino[4,3-b]quinazoline,
11-chloro-2-nitro-8H-quinazolino[4,3-b]quinazoline,
11-chloro-2-amino-8H-quinazolino[4,3-b]quinazoline,
11-chloro-2-methoxy-3-trifluoromethyl-8H-quinazolino[4,3-b]quinazoline,
11-chloro-2-(3-fluoro-benzyloxy)3-methoxy-8H-quinazolino[4,3-b]quinazoline,
9-bromo-3-methoxy-2-n-propyl-8H-quinazolino[4,3-b]quinazoline,
9-bromo-8H-quinazolino[4,3-b]quinazoline-2- formic acid ethyl ester,
(E)-(9-bromo-8H-quinazolino[4,3-b]quinazoline-2-yl)-4(dimethylamino)-2-butene amide,
9-bromo-8H-quinazolino[4,3-b]quinazoline-2-formic acid,
4-(2,3-dimethoxy-8H-quinazolino[4,3-b]quinazoline-9-yl)morpholine, and
11-chloro-2,3-dimethoxy-8-trifluoromethyl-8H-quinazolino[4,3-b]quinazoline, pharmaceutically acceptable salts thereof, and hydrates of the pharmaceutically acceptable salts.

25. A pharmaceutical composition comprising the at least one active pharmaceutical ingredient according to claim 1 and at least one pharmaceutically acceptable carrier.

26. A method for treating cancers susceptible to treatment with protein tyrosine kinase inhibitors and/or aurora kinase inhibitors comprising administering to a patient in need thereof an amount effective for treating said susceptible cancers of the at least one active pharmaceutical ingredient according to claim 1.

27. The least one active pharmaceutical ingredient according to claim 24, wherein the pharmaceutically acceptable salts are chosen from hydrochloride, hydrobromide, sulfate, acetate, lactate, tartrate, tannate, citrate, trifluoroacetate, malate, maleate, succinate, p-toluenesulfonate, and mesylate.

28. At least one active pharmaceutical ingredient chosen from:
9-chloro-2,3-bis(2-methoxyethoxy)-8H-quinazolino[4,3-b]quinazoline hydrochloride monohydrate,
9-bromo-2,3-bis(2-methoxyethoxy)-8H-quinazolino[4,3-b]quinazoline hydrochloride hemihydrate,
9-fluoro-2,3-bis(2-methoxyethoxy)-8H-quinazolino[4,3-b]quinazoline mesylate,
9-ethynyl-2,3-bis(2-methoxyethoxy)-8H-quinazolino[4,3-b]quinazoline hydrochloride trihydrate,
9-amino-2,3-bis(2-methoxyethoxy)-8H-quinazolino[4,3-b]quinazoline hydrobromide trihydrate,
mesylate-{9-nitro-2,3-bis(2-methoxyethoxy)-8H-quinazolino[4,3-b]quinazoline-11-methanol}ester hydrochloride,
11-chloro-8-methyl-2,3-bis(2-methoxyethoxy)-8H-quinazolino[4,3-b]quinazoline malate,
4-(3-((9-chloro-10-fluoro-3-methoxy-8H-quinazolino[4,3-b]quinazoline-2-yl)oxy)propyl)morpholine di-p-toluenesulfonate trihydrate, and
(E)-(9-bromo-8H-quinazolino[4,3-b]quinazoline-2-yl)-4(dimethylamino)-2-butene amide hydrochloride.
